(19) United States
(12) Patent Application Publication
(10) Pub. No.: US 2024/0207805 A1
(43) Pub. Date: Jun. 27, 2024

(54) METHODS FOR PERFORMING IN VITRO TRANSCRIPTION USING dUTP

(71) Applicant: Nutcracker Therapeutics, Inc., Emeryville, CA (US)

(72) Inventor: Hung Pin Kao, Fremont, CA (US)

(21) Appl. No.: 18/557,657

(22) PCT Filed: Apr. 26, 2022
(86) PCT No.: PCT/US22/26267

§ 371 Date: Oct. 27, 2023

Related U.S. Application Data
(60) Provisional application No. 63/182,291, filed on Apr. 30, 2021.

(51) Int. Cl. B01J 19/00; C12N 9/24; C12P 19/34

(52) U.S. Cl. CPC B01J 19/0046; C12N 9/2497; C12P 19/34; C12Y 302/02027; B01J 2219/00788; B01J 2219/00873; B01J 2219/00957; B01J 2219/00961

(57) ABSTRACT

A method includes incubation of an amplification mixture containing uracil-DNA glycosy lase enzyme (UNG enzyme), a deoxyribonucleotide triphosphate (dNTP) mixture, a DNA polymerase, a template including a sequence of interest, and at least one primer pair, inactivation of UNG enzyme in the amplification mixture, and amplification of a sequence of interest to form an in vitro transcription template. The in vitro transcription template may be used to produce a therapeutic polynucleotide.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves may also correspond to implementations of the claimed technology.

Some currently available technologies for manufacturing and formulating polynucleotide therapeutics (e.g., mRNA therapeutics, etc.) may be vulnerable to contamination by polynucleotide sequences, particularly due to aerosolization, causing amplification of an undesired contamination product.

### SUMMARY

Development of methods for minimizing contamination by unwanted polynucleotide sequences may advance the use of polynucleotide-based therapeutic modalities. Microfluidic instrumentation and processes may be used with specified nucleotides and enzymes to provide advantages in reducing the risk of contamination in microfluidic systems. Described herein are devices, systems, and methods for addressing polynucleotide contamination within a microfluidic system. Such methods and systems may be used for the manufacture and formulation of biomolecule-containing products, such as therapeutics for individualized care.

An implementation relates to a method comprising: incubating an amplification mixture comprising a uracil-DNA glycosylase enzyme (UNG enzyme), a deoxyribonucleotide triphosphate (dNTP) mixture, a DNA polymerase, a template comprising a sequence of interest, and at least one primer pair; inactivating the UNG enzyme in the amplification mixture; amplifying the sequence of interest from the template to form an in vitro transcription (IVT) template, the IVT template comprising a uracil-containing polynucleotide sequence corresponding to the sequence of interest; and performing in vitro transcription (IVT) from the IVT template to produce a therapeutic polynucleotide; the deoxyribonucleotide triphosphate (dNTP) mixture comprising a dUTP, a modified dUTP, or combinations thereof.

In some implementations of the method, such as that described in the preceding paragraph of this summary, the template comprises a DNA template.

In some implementations of the method, such as any of those described in any preceding paragraphs of this summary, the template may comprise linear DNA.

In some implementations of the method, such as any of those described in any preceding paragraphs of this summary, the template may comprise plasmid DNA.

In some implementations of the method, such as any of those described in any preceding paragraphs of this summary, the incubating may be carried out until a uracil-containing sequence in the amplification mixture is at least 90% degraded by the UNG enzyme.

In some implementations of the method, such as any of those described in any preceding paragraphs of this summary, the inactivating may be carried out until the UNG enzyme is at least 90% inactivated.

In some implementations of the method, such as any of those described in any preceding paragraphs of this summary, the inactivating may follow the incubating.

In some implementations of the method, such as any of those described in any preceding paragraphs of this summary, the inactivating may involve thermal inactivation.

In some implementations of the method, such as any of those described in any preceding paragraphs of this summary, the UNG enzyme may be cod UNG enzyme.

In some implementations of the method, such as any of those described in any preceding paragraphs of this summary, the amplification mixture may be substantially free of dTTP.

In some implementations of the method, such as any of those described in any preceding paragraphs of this summary, the amplification mixture may comprise a modified dUTP selected from a 5-substituted dUTP analog, a biotinylated dNTP, 5-modified biotin-16-aminoallyl dUTP, hydroxymethyl dUTP (“dITP”), Biotin-UTP, Digoxigenin-UTP, 2′-F-UTP, biotin-4-dUTP, biotin-11-dUTP, biotin-14-dUTP, biotin-16-AA-dUTP, 5-iodo-dUTP, 5-bromo-dUTP, 5-fluoro-dUTP, 5-propynyl-dUTP, and combinations thereof.

In some implementations of the method, such as any of those described in any preceding paragraphs of this summary, the therapeutic polynucleotide may comprise an mRNA.

In some implementations of the method, such as any of those described in any preceding paragraphs of this summary, the therapeutic polynucleotide may comprise a single-stranded mRNA.

In some implementations of the method, such as any of those described in any preceding paragraphs of this summary, the therapeutic polynucleotide may be selected from an mRNA, a circular RNA, a self-replicating RNA, or combinations thereof.

In some implementations of the method, such as any of those described in any preceding paragraphs of this summary, the method may comprise purifying a therapeutic polynucleotide.

In some implementations of the method, such as any of those described in any preceding paragraphs of this summary, the method may comprise concentrating a therapeutic polynucleotide.

In some implementations of the method, such as any of those described in any preceding paragraphs of this summary, the method may comprise combining the therapeutic polynucleotide with a delivery vehicle to form a therapeutic polynucleotide composition.

In some implementations of the method, such as any of those described in any preceding paragraphs of this summary, the therapeutic polynucleotide composition may comprise a therapeutic polynucleotide encapsulated by a delivery vehicle, said delivery vehicle comprising an amphipathic molecule.

In some implementations of the method, such as any of those described in any preceding paragraphs of this summary, the delivery vehicle may comprise a peptoid.

In some implementations of the method, such as any of those described in any preceding paragraphs of this summary, the contacting; inactivating; and amplifying may be performed in a first chamber of a process chip.

In some implementations of the method, such as any of those described in any preceding paragraphs of this summary, the act of performing in vitro transcription (IVT) may be performed in a second chamber of the process chip.

An implementation relates to a method comprising: amplifying a sequence of interest from a template to form an IVT template comprising a uracil-containing polynucleotide sequence corresponding to the sequence of interest; and performing in vitro transcription from the IVT template to produce a therapeutic polynucleotide.

In some implementations of the method, such as any of those described in any preceding paragraphs of this summary, the template may comprise a DNA template.

In some implementations of the method, such as any of those described in any preceding paragraphs of this summary, the template may comprise linear DNA.

In some implementations of the method, such as any of those described in any preceding paragraphs of this summary, the template may comprise plasmid DNA.

In some implementations of the method, such as any of those described in any preceding paragraphs of this summary, the amplifying may be carried out in an amplification mixture, the amplification mixture being substantially free of dTTP.

In some implementations of the method, such as any of those described in any preceding paragraphs of this summary, the amplifying may be carried out in an amplification mixture comprising UNG enzyme.

In some implementations of the method, such as any of those described in any preceding paragraphs of this summary, the UNG enzyme may be Cod UNG enzyme.

In some implementations of the method, such as any of those described in any preceding paragraphs of this summary, the amplifying may be carried out in an amplification mixture comprising a modified dUTP selected from a 5-substituted dUTP analog, a biotinylated dNTP, 5-modified biotin-16-aminoallyl dUTP, hydroxymethyl dUTP (“dITP”), Biotin-UTP, Digoxigenin-UTP, 2′-F-UTP, biotin-4-dUTP, biotin-11-dUTP, biotin-14-dUTP, biotin-16-AA-dUTP, 5-iodo-dUTP, 5-bromo-dUTP, 5-fluoro-dUTP, 5-propynyl-dUTP, and combinations thereof.

In some implementations of the method, such as any of those described in any preceding paragraphs of this summary, the therapeutic polynucleotide may comprise an mRNA.

In some implementations of the method, such as any of those described in any preceding paragraphs of this summary, the therapeutic polynucleotide may comprise a single-stranded mRNA.

In some implementations of the method, such as any of those described in any preceding paragraphs of this summary, the therapeutic polynucleotide may be selected from an mRNA, a circular RNA, a self-replicating RNA, or combinations thereof.

In some implementations of the method, such as any of those described in any preceding paragraphs of this summary, the method may comprise purifying the therapeutic polynucleotide.

In some implementations of the method, such as any of those described in any preceding paragraphs of this summary, the method may further comprise concentrating the therapeutic polynucleotide.

In some implementations of the method, such as any of those described in any preceding paragraphs of this summary, the method may further comprise combining the therapeutic polynucleotide with a delivery vehicle to form a therapeutic polynucleotide composition.

In some implementations of the method, such as any of those described in any preceding paragraphs of this summary, the therapeutic polynucleotide composition may comprise the therapeutic polynucleotide encapsulated by the delivery vehicle, said delivery vehicle comprising an amphipathic molecule.

In some implementations of the method, such as any of those described in any preceding paragraphs of this summary, the delivery vehicle may comprise a peptoid.

In some implementations of the method, such as any of those described in any preceding paragraphs of this summary, the amplifying may be performed in a first chamber of a process chip.

In some implementations of the method, such as any of those described in any preceding paragraphs of this summary, the act of performing in vitro transcription (IVT) may be performed in a second chamber of the process chip.

An implementation relates to a composition comprising an amplification mixture and uracil-DNA glycosylase (UNG) enzyme, the amplification mixture comprising a DNA polymerase, at least one primer pair, a template comprising a sequence of interest; and a dNTP selected from dUTP, a modified dUTP, and combinations thereof.

In some implementations of the method, such as any of those described in any preceding paragraphs of this summary, the UNG enzyme may be Cod UNG enzyme.

In some implementations of the method, such as any of those described in any preceding paragraphs of this summary, the template may comprise a DNA template.

In some implementations of the method, such as any of those described in any preceding paragraphs of this summary, the template may comprise linear DNA.

In some implementations of the method, such as any of those described in any preceding paragraphs of this summary, the template may comprise plasmid DNA.

In some implementations of the method, such as any of those described in any preceding paragraphs of this summary, the amplification mixture may be substantially free of dTTP.

In some implementations of the method, such as any of those described in any preceding paragraphs of this summary, the modified dUTP may be selected from a 5-substituted dUTP analog, a biotinylated dNTP, 5-modified biotin-16-aminoallyl dUTP, hydroxymethyl dUTP (“dITP”), Biotin-UTP, Digoxigenin-UTP, 2′-F-UTP, biotin-4-dUTP, biotin-11-dUTP, biotin-14-dUTP, biotin-16-AA-dUTP, 5-iodo-dUTP, 5-bromo-dUTP, 5-fluoro-dUTP, 5-propynyl-dUTP, and combinations thereof.

An implantation relates to a therapeutic polynucleotide made using a method described in any preceding claim.

An implementation relates to an apparatus comprising: a first chamber to: incubate a UNG enzyme with an amplification mixture comprising a deoxyribonucleotide triphosphate (dNTP) mixture, a DNA polymerase, a template, and at least one primer pair; inactivate the UNG enzyme in the amplification mixture; amplify a polynucleotide sequence of interest from the template to form a uracil-containing polynucleotide; and a second chamber to: perform in vitro transcription (IVT) from the uracil-containing polynucleotide to produce a therapeutic polynucleotide.

In some implementations of the apparatus, such as any of those described in any preceding paragraphs of this summary, the apparatus may be part of a process chip.

In some implementations of the apparatus, such as any of those described in any preceding paragraphs of this summary, the first chamber and second chamber may be part of one process chip.

In some implementations of the apparatus, such as any of those described in any preceding paragraphs of this summary, the template may comprise a DNA template.

In some implementations of the apparatus, such as any of those described in any preceding paragraphs of this summary, the template may comprise linear DNA.

In some implementations of the apparatus, such as any of those described in any preceding paragraphs of this summary, the template may comprise plasmid DNA.

In some implementations of the apparatus, such as any of those described in any preceding paragraphs of this summary, the apparatus may comprise a controller to drive communication of fluid through the one or more chambers to thereby incubate an amplification mixture until a uracil-containing sequence in the amplification mixture is at least 90% degraded by the UNG enzyme.

In some implementations of the apparatus, such as any of those described in any preceding paragraphs of this summary, the apparatus may comprise a controller to drive communication of fluid through the one or more chambers to thereby inactivate at least 90% of the UNG enzyme.

In some implementations of the apparatus, such as any of those described in any preceding paragraphs of this summary, the apparatus may comprise a controller to drive communication of fluid through the one or more chambers such that the inactivating follows the incubating.

In some implementations of the apparatus, such as any of those described in any preceding paragraphs of this summary, the inactivating may involve thermal inactivation.

In some implementations of the apparatus, such as any of those described in any preceding paragraphs of this summary, the UNG enzyme may be cod UNG enzyme.

In some implementations of the apparatus, such as any of those described in any preceding paragraphs of this summary, the amplification mixture may be substantially free of dTTP.

In some implementations of the apparatus, such as any of those described in any preceding paragraphs of this summary, the amplification mixture may comprise a modified dUTP selected from a 5-substituted dUTP analog, a biotinylated dNTP, 5-modified biotin-16-aminoallyl dUTP, hydroxymethyl dUTP (“dITP”), Biotin-UTP, Digoxigenin-UTP, 2′-F-UTP, biotin-4-dUTP, biotin-11-dUTP, biotin-14-dUTP, biotin-16-AA-dUTP, 5-iodo-dUTP, 5-bromo-dUTP, 5-fluoro-dUTP, 5-propynyl-dUTP, and combinations thereof.

In some implementations of the apparatus, such as any of those described in any preceding paragraphs of this summary, the therapeutic polynucleotide may comprise an mRNA.

In some implementations of the apparatus, such as any of those described in any preceding paragraphs of this summary, the therapeutic polynucleotide may comprise a single-stranded mRNA.

In some implementations of the apparatus, such as any of those described in any preceding paragraphs of this summary, the therapeutic polynucleotide may be selected from an mRNA, a circular RNA, a self-replicating RNA, or combinations thereof.

In some implementations of the apparatus, such as any of those described in any preceding paragraphs of this summary, the apparatus may comprise a controller to drive purification of the therapeutic polynucleotide.

In some implementations of the apparatus, such as any of those described in any preceding paragraphs of this summary, the apparatus may comprise a controller to drive concentrating the therapeutic polynucleotide.

In some implementations of the apparatus, such as any of those described in any preceding paragraphs of this summary, the apparatus may comprise a controller to drive combining of the therapeutic polynucleotide with a delivery vehicle to form a therapeutic polynucleotide composition.

In some implementations of the apparatus, such as any of those described in any preceding paragraphs of this summary, the therapeutic polynucleotide composition comprising the therapeutic polynucleotide encapsulated by the delivery vehicle. The delivery vehicle may comprise an amphipathic molecule.

In some implementations of the apparatus, such as any of those described in any preceding paragraphs of this summary, the delivery vehicle may comprise a peptoid.

An implementation relates to a system comprising: one apparatus comprising one or more chambers, the apparatus removably inserted into the system; and a controller to drive communication of fluid through the one or more chambers to thereby: incubate uracil-DNA glycosylase (UNG) enzyme with an amplification mixture comprising a deoxyribonucleotide triphosphate (dNTP) mixture, a DNA polymerase, a template material, and at least one primer pair; inactivate the UNG enzyme in the amplification mixture; amplify a polynucleotide sequence of interest from the template precursor material to form a uracil-containing polynucleotide; and perform in vitro transcription (IVT) from the uracil-containing polynucleotide to produce a therapeutic polynucleotide.

In some implementations of the system, such as any of those described in any preceding paragraphs of this summary, the apparatus may be part of a process chip.

In some implementations of the system, such as any of those described in any preceding paragraphs of this summary, the one or more chambers may be part of one process chip.

In some implementations of the system, such as any of those described in any preceding paragraphs of this summary, the template may comprise a DNA template.

In some implementations of the system, such as any of those described in any preceding paragraphs of this summary, the template may comprise linear DNA.

In some implementations of the system, such as any of those described in any preceding paragraphs of this summary, the template may comprise plasmid DNA.

In some implementations of the system, such as any of those described in any preceding paragraphs of this summary, the controller to drive communication of fluid through the one or more chambers to thereby incubate uracil-DNA glycosylase (UNG) enzyme with the amplification mixture until a uracil-containing sequence in said amplification mixture may be at least 90% degraded by said UNG enzyme

In some implementations of the system, such as any of those described in any preceding paragraphs of this summary, the controller to drive communication of fluid through the one or more chambers to thereby inactivate said UNG enzyme in said amplification mixture until at least 90% of said UNG enzyme is inactivated.

In some implementations of the system, such as any of those described in any preceding paragraphs of this summary, the controller to drive communication of fluid through the one or more chambers to thereby inactivate said UNG enzyme in said amplification mixture after incubating uracil-DNA glycosylase (UNG) enzyme with the amplification mixture.

In some implementations of the system, such as any of those described in any preceding paragraphs of this summary, the said controller to drive communication of fluid through the one or more chambers to thereby inactivate said UNG enzyme in the amplification mixture through thermal inactivation.

In some implementations of the system, such as any of those described in any preceding paragraphs of this summary, the UNG enzyme may be cod UNG enzyme.

In some implementations of the system, such as any of those described in any preceding paragraphs of this summary, the amplification mixture may be substantially free of dTTP.

In some implementations of the system, such as any of those described in any preceding paragraphs of this summary, the amplification mixture may comprise a modified dUTP selected from a 5-substituted dUTP analog, a biotinylated dNTP, 5-modified biotin-16-aminoallyl dUTP, hydroxymethyl dUTP (“dITP”), Biotin-UTP, Digoxigenin-UTP, 2′-F-UTP, biotin-4-dUTP, biotin-11-dUTP, biotin-14-dUTP, biotin-16-AA-dUTP, 5-iodo-dUTP, 5-bromo-dUTP, 5-fluoro-dUTP, 5-propynyl-dUTP, and combinations thereof.

In some implementations of the system, such as any of those described in any preceding paragraphs of this summary, the therapeutic polynucleotide may comprise an mRNA.

In some implementations of the system, such as any of those described in any preceding paragraphs of this summary, the therapeutic polynucleotide may comprise a single-stranded mRNA.

In some implementations of the system, such as any of those described in any preceding paragraphs of this summary, the therapeutic polynucleotide may be selected from an mRNA, a circular RNA, a self-replicating RNA, or combinations thereof.

In some implementations of the system, such as any of those described in any preceding paragraphs of this summary, the controller to drive communication of fluid through the one or more chambers to thereby purify the therapeutic polynucleotide.

In some implementations of the system, such as any of those described in any preceding paragraphs of this summary, the controller to drive communication of fluid through the one or more chambers to thereby concentrate the therapeutic polynucleotide.

In some implementations of the system, such as any of those described in any preceding paragraphs of this summary, the controller to drive communication of fluid through the one or more chambers to thereby combine the therapeutic polynucleotide with a delivery vehicle to form a therapeutic polynucleotide composition.

In some implementations of the system, such as any of those described in any preceding paragraphs of this summary, the therapeutic polynucleotide composition comprising the therapeutic polynucleotide encapsulated by the delivery vehicle. The delivery vehicle may comprise an amphipathic molecule.

In some implementations of the system, such as any of those described in any preceding paragraphs of this summary, the delivery vehicle may comprise a peptoid.

It should be appreciated that all combinations of the foregoing concepts and additional concepts discussed in greater detail below (provided such concepts are not mutually inconsistent) are contemplated as being part of the inventive subject matter disclosed herein and to achieve the benefits as described herein.

### BRIEF DESCRIPTION OF THE DRAWINGS

The details of one or more implementations are set forth in the accompanying drawings and the description below. Other features, aspects, and advantages will become apparent from the description, the drawings, and the claims, in which:

FIG. 1 depicts a schematic view of an example of a microfluidic system;

FIG. 2 depicts an exploded perspective view of examples of components of the system of FIG. 1;

FIG. 3 depicts a top plan view of an example of a process chip that may be incorporated into the system of FIG. 1;

FIG. 4 is a section through a portion of one variation of an example process chip;

FIG. 5 schematically illustrates one variation of an example method of manufacturing an mRNA therapeutic;

FIG. 6 depicts an example of a double-stranded DNA template described in some examples herein;

FIG. 7 depicts an example of a functional diagram for a process chip configured to perform IVT;

FIG. 8 schematically illustrates an example of a functional diagram for a process chip configured as a formulation process chip; and

FIG. 9 schematically illustrates an example of a functional diagram for a process chip configured as a formulation process chip.

### DETAILED DESCRIPTION

In some aspects, apparatuses and methods are disclosed herein for processing therapeutic polynucleotides. In particular, these apparatuses and methods may be closed path apparatuses and methods that are configured to minimize or eliminate manual handling during operation. The closed path apparatuses and methods may provide a nearly entirely aseptic environment, and the components may provide a sterile path for processing from initial input (e.g., template) to output (e.g., compounded therapeutic). Material inputs (e.g., nucleotides, and any chemical components) into the apparatus may be sterile; and may be introduced into the system without requiring virtually any manual interaction.

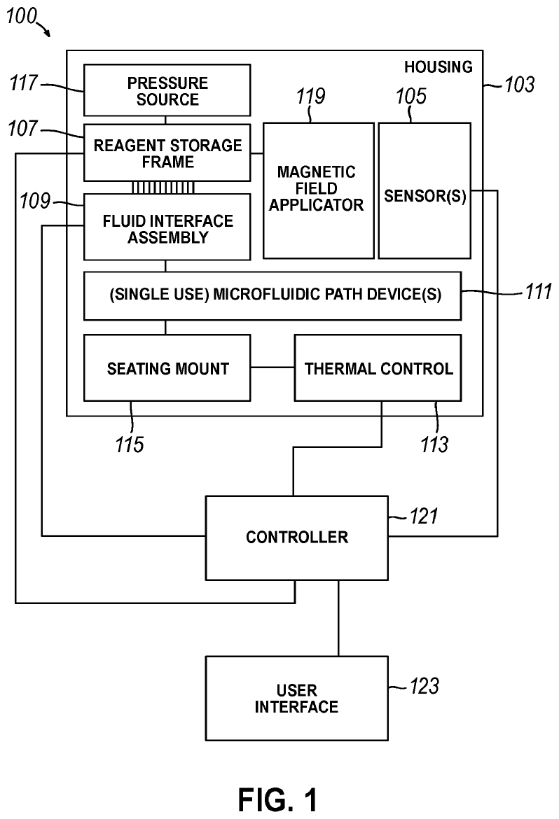

The apparatuses and methods described herein may be used to generate therapeutics at rapid cycle times at high degree of reproducibility. The apparatuses described herein may be configured to provide, in a single integrated apparatus, synthesis, purification, dialysis, compounding, and concentration of one or more therapeutic compositions. Alternatively, one or more of these processes may be carried out in two or more apparatuses as described herein. In some scenarios, the therapeutic compositions may include therapeutic polynucleotides, such as, for example, ribonucleic acids or deoxyribonucleic acids. The polynucleotides may include only natural nucleotide units or may include any kind of synthetic, semi-synthetic, or modified nucleotide units. All or some of the processing steps may be performed in an unbroken fluid processing pathway, which may be configured as one or a series of consumable microfluidic path device(s)—in some instances hereinsometimes also referred to as a process chip or a biochip (though the chip need not necessarily be used in bio-related applications). The apparatus in in some examples may be removably installed in an instrument that is part of a larger microfluidic system, such as that shown in FIG. 1). The disclosed apparatuses and methods may be used for the synthesis of patient-specific therapeutics, including compounding, at a point of care (e.g. hospital, clinic, pharmacy, etc.).

The disclosed apparatuses and methods may employ amplification of polynucleotides to generate a polynucleotide therapeutic. Because of the sensitivity of many amplification methods, such as polymerase chain reaction (PCR), including quantitative real-time polymerase chain reaction (qPCR), digital polymerase chain reaction (dPCR), and next-generation sequencing (NGS), even a small amount of a product of an amplification reaction into a laboratory area or device may lead to contamination, frustrating accurate amplification of other samples. The disclosed apparatuses and methods allow for synthesis, purification, dialysis, compounding, and concentration of one or more therapeutic compositions with reduced risk of contamination by nucleotide amplification products.

### Terminology

Throughout this specification and the claims which follow, unless the context requires otherwise, the word “comprise”, and variations such as “comprises” and “comprising” means various components may be co-jointly employed in the methods and articles (e.g., compositions and apparatuses including device and methods). For example, the term “comprising” will be understood to imply the inclusion of any stated elements or steps but not the exclusion of any other elements or steps. In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is ±0.1% of the stated value (or range of values), ±1% of the stated value (or range of values), ±2% of the stated value (or range of values), ±5% of the stated value (or range of values), ±10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein.

It is also understood that when a value is disclosed that “less than or equal to” the value, “greater than or equal to the value,” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although the terms “first” and “second” may be used herein to describe various features/elements (including steps), these features/elements should not be limited by these terms, unless the context indicates otherwise. These terms are used to distinguish one feature/element from another feature/element, and unless specifically pointed out, do not denote a certain order. Thus, a first feature/element discussed below could be termed a second feature/element, and similarly, a second feature/element discussed below could be termed a first feature/element without departing from the teachings of the present invention.

As used herein, “polynucleotide” refers to a nucleic acid molecule containing multiple nucleotides and generally refers both to “oligonucleotides” (a polynucleotide molecule of 18-25 nucleotides in length) and polynucleotides of 26 or more nucleotides. Aspects of this disclosure include compositions including oligonucleotides having a length of 18-25 nucleotides (e. g., 18-mers, 19-mers, 20-mers, 21-mers, 22-mers, 23-mers, 24-mers, or 25-mers), or medium-length polynucleotides having a length of 26 or more nucleotides (e.g., polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length. Where a polynucleotide is double-stranded, its length may be similarly described in terms of base pairs.

As used herein “amplification” may refer to polynucleotide amplification. Amplification may include any suitable method for amplification of a polynucleotide and includes, but is not limited to, multiple displacement amplification (MDA), polymerase chain reaction (PCR) amplification, Loop Mediated Isothermal Amplification (LAMP), Nucleic Acid Sequence Based Amplification, Strand Displacement Amplification, Rolling Circle Amplification, and Ligase Chain Reaction.

As used herein a “cassette” (e.g., a synthetic in vitro transcription facilitator cassette) refers to a polynucleotide sequence which may include or be operably linked to one or more expression elements such as an enhancer, a promoter, a leader, an intron, a 5′ untranslated region (UTR), a 3′ UTR, or a transcription termination sequence. In some aspects, a cassette comprises at least a first polynucleotide sequence capable of initiating transcription of an operably linked second polynucleotide sequence (which may comprise a template) and optionally a transcription termination sequence operably linked to the second polynucleotide sequence. The template, as described below, may comprise a sequence of interest, for example, an open reading frame (“ORF”) of interest. The cassette may be provided as a single element or as two or more unlinked elements.

As used herein, a “template” refers to a nucleic acid sequence that contains a sequence of interest for preparing a therapeutic polynucleotide according to the disclosed methods. Templates may be, but are not limited to, a double stranded DNA (dsDNA), an engineered plasmid construct, a cDNA sequence, or a linear nucleic acid sequence (for example, a linear template generated by PCR or by annealing chemically synthesized oligonucleotides). The template may, in certain aspects, be integrated into a “cassette” as described above.

As used herein, the term “sequence of interest” refers to a polynucleotide sequence, the use of which may be deemed desirable for a suitable purpose, in particular, for the manufacture of an mRNA for a therapeutic use, and includes but is not limited to, coding sequences of structural genes, and non-coding regulatory sequences that do not encode and mRNA or protein product.

As used herein, “in vitro transcription” or “IVT” refer to the process whereby transcription occurs in vitro in a non-cellular system to produce synthetic RNA molecules (e.g., synthetic mRNA) for use in various applications, including for therapeutic delivery to a subject, for example, as a therapeutic polynucleotide, which may be part of, or may be used to form, a therapeutic polynucleotide composition as described below. The therapeutic polynucleotide, (e.g., synthetic RNA molecules (transcription product)) generated may be combined with a delivery vehicle to form a therapeutic polynucleotide composition. Synthetic transcription products include mRNAs, antisense RNA molecules, shRNA, circular RNA molecules, ribozymes, and the like. An IVT reaction may use a purified linear DNA template comprising a promoter sequence and the sequence of the open reading frame (ORF) of a sequence of interest, ribonucleotide triphosphates or modified ribonucleotide triphosphates, a buffer system that includes DTT and magnesium ions, and a phage RNA polymerase.

As used herein a “therapeutic polynucleotide” refers to a polynucleotide (e.g., an mRNA) that may be part of a therapeutic polynucleotide composition for delivery to a subject to treat a symptom, disease, or condition in a subject; prevent a symptom, disease, or condition in a subject; or to improve or otherwise modify the subject's health.

As used herein a “therapeutic polynucleotide composition” (or “therapeutic composition” for short) may refer to a composition including one or more therapeutic polynucleotide (e.g., mRNA) encapsulated by a delivery vehicle, which composition may be administered to a subject in need thereof using any suitable administration routes, such as intratumoral, intramuscular, etc. injection. An example of a therapeutic polynucleotide composition is a mRNA nanoparticle comprising at least one mRNA encapsulated by a delivery vehicle molecule (or “delivery vehicle” for short). An mRNA vaccine is one example of a therapeutic polynucleotide composition.

As used herein, “delivery vehicle” refers to any substance that facilitates, at least in part, the in vivo, in vitro, or ex vivo delivery of a polynucleotide (e.g., therapeutic polynucleotide) to targeted cells or tissues (e.g., tumors, etc.). Referring to something as a delivery vehicle need not exclude the possibility of the delivery vehicle also having therapeutic effects. Some versions of a delivery vehicle may provide additional therapeutic effects. In some versions, a delivery vehicle may be a peptoid molecule, such as an amino-lipidated peptoid molecule, that may be used to at least partially encapsulate mRNA.

As used herein, “joining” refers to methods such as ligation, synthesis, primer extension, annealing, recombination, or hybridization use to couple one component to another.

As used herein “purifying” refers to physical and/or chemical separation of a component (e.g., particles) of other unwanted components (e.g., contaminating substances, fragments, etc.).

### I. Overview of System Including Microfluidic Process Chip

FIG. 1 depicts examples of various components that may be incorporated into a system (100). System (100) of this example includes a housing (103) enclosing a seating mount (115) that may removably hold one or more microfluidic process chips (111). In other words, system (100) includes a component that is configured to removably accommodate a process chip (111), where the process chip (111) itself defines one or more microfluidic channels or fluid pathways. Components of system (100) (e.g., within housing (103)) that fluidically interact with process chip (111) may include fluid channels or pathways that are not necessarily considered microfluidic (e.g., with such fluid channels or pathways being larger than the microfluidic channels or fluid pathways in process chip (111)). In some versions, process chips (111) are provided and utilized as single-use devices, while the rest of system (100) is reusable. Housing (103) may be in the form of a chamber, enclosure, etc., with an opening that may be closed (e.g., via a lid or door, etc.) to thereby seal the interior. Housing (103) may enclose a thermal regulator and/or may be configured to be enclosed in a thermally-regulated environment (e.g., a refrigeration unit, etc.). Housing (103) may form an aseptic barrier. In some variations, housing (103) may form a humidified or humidity-controlled environment. In addition, or in the alternative, system (100) may be positioned in a cabinet (not shown). Such a cabinet may provide a temperature-regulated (e.g., refrigerated) environment. Such a cabinet may also provide air filtering and air flow management and may promote reagents being kept at a desired temperature through the manufacturing process. In addition, such a cabinet may be equipped with UV lamps for sterilization of process chip (111) and other components of system (100). Other suitable features that may be incorporated into a cabinet that houses system (100). Seating mount (115) may be configured to secure process chip (111) using one or more pins or other components configured to hold process chip (111) in a fixed and predefined orientation. Seating mount (115) may thus facilitate process chip (111) being held at an appropriate position and orientation in relation to other components of system (100). In the present example, seating mount (115) is configured to hold process chip (111) in a horizontal orientation, such that process chip (111) is parallel with the ground.

In some variations, a thermal control (113) may be located adjacent to seating mount (115), to modulate the temperature of any process chip (111) mounted in seating mount (115). Thermal control (113) may include a thermoelectric component (e.g., Peltier device, etc.) and/or one or more heat sinks for controlling the temperature of all or a portion of any process chip (111) mounted in seating mount (115). In some variations, more than one thermal control (113) may be included, such as to separately regulate the temperature of different ones of one or more regions of process chip (111). Thermal control (113) may include one or more thermal sensors (e.g., thermocouples, etc.) that may be used for feedback control of process chip (111) and/or thermal control (113).

As shown in FIG. 1, a fluid interface assembly (109) couples process chip (111) with a pressure source (117), thereby providing one or more paths for fluid (e.g., gas) at a positive or negative pressure to be communicated from pressure source (117) to one or more interior regions of process chip (111) as will be described in greater detail below. While only one pressure source (117) is shown, system (100) may include two or more pressure sources (117). In some scenarios, pressure may be generated by one or more sources other than pressure source (117). For instance, one or more vials or other fluid sources within reagent storage frame (107) may be pressurized. In addition, or in the alternative, reactions and/or other processes carried out on process chip (111) may generate additional fluid pressure. In the present example, fluid interface assembly (109) also couples process chip (111) with a reagent storage frame (107), thereby providing one or more paths for liquid reagents, etc., to be communicated from reagent storage frame (107) to one or more interior regions of process chip (111) as will be described in greater detail below.

In some versions, pressurized fluid (e.g., gas) from at least one pressure source (117) reaches fluid interface assembly (109) via reagent storage frame (107), such that reagent storage frame (107) includes one or more components interposed in the fluid path between pressure source (117) and fluid interface assembly (109). In some versions, one or more pressure sources (117) are directly coupled with fluid interface assembly, such that the positively pressurized fluid (e.g., positively pressurized gas) or negatively pressurized fluid (e.g., suction or other negatively pressurized gas) bypasses reagent storage frame (107) to reach fluid interface assembly (109). Regardless of whether the fluid interface assembly (109) is interposed in the fluid path between pressure source (117) and fluid interface assembly (109), fluid interface assembly (109) may be removably coupled to the rest of system (100), such that at least a portion of fluid interface assembly (109) may be removed for sterilization between uses. As described in greater detail below, pressure source (117) may selectively pressurize one or more chamber regions on process chip (111). In addition, or in the alternative, pressure source may also selectively pressurize one or more vials or other fluid storage containers held by reagent storage frame (107).

Reagent storage frame (107) is configured to contain a plurality of fluid sample holders, each of which may hold a fluid vial that is configured to hold a reagent (e.g., nucleotides, solvent, water, etc.) for delivery to process chip (111). In some versions, one or more fluid vials, or other storage containers in reagent storage frame (107) may be configured to receive a product from the interior of the process chip (111). In addition, or in the alternative, a second process chip (111) may receive a product from the interior of a first process chip (111), such that one or more fluids are transferred from one process chip (111) to another process chip (111). In some such scenarios, the first process chip (111) may perform a first dedicated function (e.g., synthesis, etc.) while the second process chip (111) performs a second dedicated function (e.g., encapsulation, etc.). Reagent storage frame (107) of the present example includes a plurality of pressure lines and/or a manifold configured to divide one or more pressure sources (117) into a plurality of pressure lines that may be applied to process chip (111). Such pressure lines may be independently or collectively (in sub-combinations) controlled.

Fluid interface assembly (109) may include a plurality of fluid lines and/or pressure lines where each such line includes a biased (e.g., spring-loaded) holder or tip that individually and independently drives each fluid and/or pressure line to process chip (111) when process chip (111) is held in seating mount (115). Any associated tubing (e.g., the fluid lines and/or the pressure lines) may be part of fluid interface assembly (109) and/or may connect to fluid interface assembly (109). In some versions, each fluid line comprises a flexible tubing that connects between reagent storage frame (107), via a connector that couples the vial to the tubing in a locking engagement (e.g., ferrule) and process chip (111). In some versions, the ends of the fluid lines/pressure lines, may be configured to seal against process chip (111), e.g., at a corresponding sealing port formed in process chip (111), as described below. In the present example, the connections between pressure source (117) and process chip (111), and the connections between vials in reagent storage frame (107) and process chip (111), all form sealed and closed paths that are isolated when process chip (111) is seated in seating mount (115). Such sealed, closed paths may provide protection against contamination when processing therapeutic polynucleotides.

The vials of reagent storage frame (107) may be pressurized (e.g., >1 atm pressure, such as 2 atm, 3 atm, 5 atm, or higher). In some versions, the vials may be pressurized by pressure source (117). Negative or positive pressure may thus be applied. For example, the fluid vials may be pressurized to between about 1 and about 20 psig (e.g., 5 psig, 10 psig, etc.). Alternatively, a vacuum (e.g., about −7 psig or about 7 psia) may be applied to draw fluids back into the vials (e.g., vials serving as storage depots) at the end of the process. The fluid vials may be driven at lower pressure than the pneumatic valves as described below, which may prevent or reduce leakage. In some variations, the difference in pressure between the fluid and pneumatic valves may be between about 1 psi and about 25 psi (e.g., about 3 psi, about 5 psi, 7 psi, 10 psi, 12 psi, 15 psi, 20 psi, etc.).

System (100) of the present example further includes a magnetic field applicator (119), which is configured to create a magnetic field at a region of the process chip (111). Magnetic field applicator (119) may include a movable head that is operable to move the magnetic field to thereby selectively isolate products that are adhered to magnetic capture beads within vials or other storage containers in reagent storage frame (107).

System (100) of the present example further includes one or more sensors (105). In some versions, such sensors (105) include one or more cameras and/or other kinds of optical sensors. Such sensors (105) may sense one or more of a barcode, a fluid level within a fluid vial held within reagent storage frame (107), fluidic movement within a process chip (111) that is mounted within seating mount (115), and/or other optically detectable conditions. In versions where a sensor (105) is used to sense barcodes, such barcodes may be included on vials of reagent storage frame (107), such that sensor (105) may be used to identify vials in reagent storage frame (107). In some versions, a single sensor (105) is positioned and configured to simultaneously view such barcodes on vials in reagent storage frame (107), fluid levels in vials in reagent storage frame (107), fluidic movement within a process chip (111) that is mounted within seating mount (115), and/or other optically detectable conditions. In some other versions, more than one sensor (105) is used to view such conditions. In some such versions, different sensors (105) may be positioned and configured to separately view corresponding optically detectable conditions, such that a sensor (105) may be dedicated to a particular corresponding optically detectable condition.

In versions where sensors (105) include at least one optical sensor, visual/optical markers may be used to estimate yield. For example, fluorescence may be used to detect process yield or residual material by tagging with fluorophores. In addition, or in the alternative, dynamic light scattering (DLS) may be used to measure particle size distributions within a portion of the process chip (111) (e.g., such as a mixing portion of process chip (111)). In some variations, sensor (105) may provide measurements using one or two optical fibers to convey light (e.g., laser light) into process chip (111); and detect an optical signal coming out of process chip (111). In versions where sensor (105) optically detects process yield or residual material, etc., sensor (105) may be configured to detect visible light, fluorescent light, an ultraviolet (UV) absorbance signal, an infrared (IR) absorbance signal, and/or any other suitable kind of optical feedback.

In versions where sensors (105) include at least one optical sensor that is configured to capture video images, such sensors (105) may record at least some activity on process chip (111). For example, an entire run for synthesizing and/or processing a material (e.g., a therapeutic RNA) may be recorded by one or more video sensors (105), including a video sensor (105) that may visualize process chip (111) (e.g., from above). Processing on process chip (111) may be visually tracked and this video record may be retained for later quality control and/or processing. Thus, the video record of the processing may be saved, stored, and/or transmitted for subsequent review and/or analysis. In addition, as will be described in greater detail below, the video may be used as a real-time feedback input that may affect processing using at least visually observable conditions captured in the video.

System (100) may be controlled by a controller (121). Controller (121) may include one or more processors, one or more memories, and various other suitable electrical components. In some versions, one or more components of controller (121) (e.g., one or more processors, etc.) is/are embedded within system (100) (e.g., contained within housing (103)). In addition, or in the alternative, one or more components of controller (121) (e.g., one or more processors, etc.) may be detachably attached or detachably connected with other components of system (100). Thus, at least a portion of controller (121) may be removable. Moreover, at least a portion of controller (121) may be remote from housing (103) in some versions.

The control by controller (121) may include activating pressure source (117) to apply pressure through process chip (111) to drive fluidic movement, among other tasks. Controller (121) may be completely or partially outside of housing (103); or completely or partially inside of housing (103). Controller (121) may be configured to receive user inputs via a user interface (123) of system (100); and provide outputs to users via user interface (123). In some versions, controller (121) is fully automated to a point where user inputs are not needed. In some such versions, user interface (123) may provide only outputs to users. User interface (123) may include a monitor, a touchscreen, a keyboard, and/or any other suitable features. Controller (121) may coordinate processing, including moving one or more fluid(s) onto and on process chip (111), mixing one or more fluids on process chip (111), adding one or more components to process chip (111), metering fluid in process chip (111), regulating the temperature of process chip (111), applying a magnetic field (e.g., when using magnetic beads), etc. Controller (121) may receive real-time feedback from sensors (105) and execute control algorithms in accordance with such feedback from sensors (105). Such feedback from sensors (105) may include, but need not be limited to, identification of reagents in vials in reagent storage frame (107), detected fluid levels in vials in reagent storage frame (107), detected movement of fluid in process chip (111), fluorescence of fluorophores in fluid in process chip (111), etc. Controller (121) may include software, firmware and/or hardware. Controller (121) may also communicate with a remote server, e.g., to track operation of the apparatus, to re-order materials (e.g., components such as nucleotides, process chips (111), etc.), and/or to download protocols, etc.

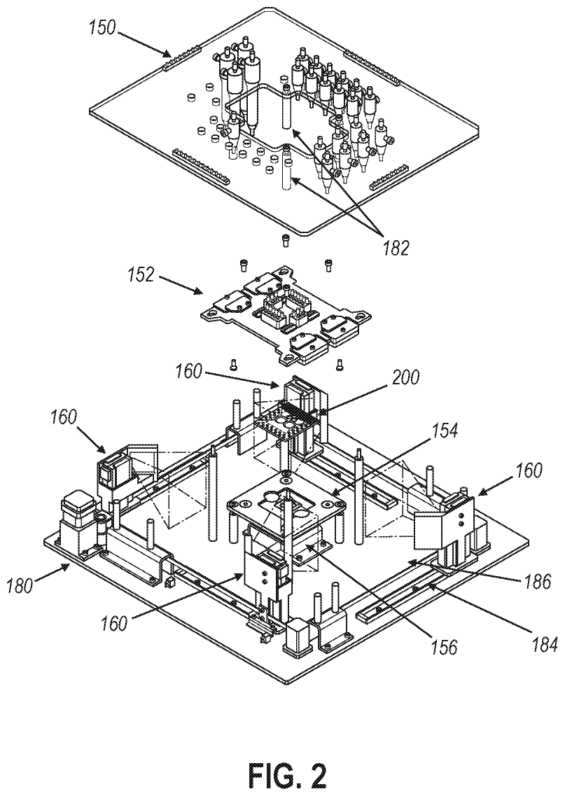

FIG. 2 shows examples of certain forms that may be taken by various components of system (100). In particular, FIG. 2 shows a reagent storage frame (150), a fluid interface assembly (152), a seating mount (154), a thermal control (156), and a process chip (200). Reagent storage frame (150), fluid interface assembly (152), seating mount (154), thermal control (156), and process chip (200) of this example may be configured and operable just like reagent storage frame (107), fluid interface assembly (109), seating mount (115), thermal control (113), and process chip (111), respectively, described above. These components are secured relative to a base (180). A set of rods (182) support reagent storage frame (150) over fluid interface assembly (152).

As shown in FIG. 2, a set of optical sensors (160) are positioned at four respective locations along base (180). Optical sensors (160) may be configured and operable like sensors (105) described above. Optical sensors (160) may include off-the-shelf cameras or any other suitable kinds of optical sensors. Optical sensors (160) are positioned such that fluid vials held within reagent storage frame (150) are within the field of view of one or more of optical sensors (160). In addition, process chip (200) is within the field of view of one or more of optical sensors (160). Each optical sensor (160) is movably secured to base (180) via a corresponding rail (184) (e.g., in a gantry arrangement), such that each optical sensor (160) is configured to translate laterally along each corresponding rail (184). A linear actuator (186) is secured to each optical sensor (160) and is thereby operable to drive lateral translation of each optical sensor (160) along the corresponding rail (184). Each actuator (186) may be in the form of a drive belt, a drive chain, a drive cable, or any other suitable kind of structure. Controller (121) may drive operation of actuators (186). Optical sensors (160) may be moved along rails (184) during operation of system (100) in order to facilitate viewing of the appropriate regions of vials in reagent storage frame (150) and/or process chip (200). In some scenarios, optical sensors (160) move in unison along corresponding rails (184). In some other scenarios, optical sensors (160) move independently along corresponding rails (184).

While optical sensors (160) are shown in FIG. 2 as being mounted to base (180), optical sensors (160) may be positioned elsewhere within system (100), in addition to or as an alternative to being mounted to base (180). For instance, some versions of reagent storage frame (107) may include one or more optical sensors (160) positioned and configured to provide an overhead field of view. In some such versions, such optical sensors (160) may be mounted to rails, movable cantilever arms, or other structures that allow such optical sensors (160) to be repositioned during operation of system (100). Other suitable locations in which optical sensors (160) may be positioned may be used. While not shown, system (100) may also include one or more sources of light (e.g., electroluminescent panels, etc.) to provide illumination that aids in optical sensing by optical sensors (160).

In some versions, one or more mirrors are used to facilitate visualization of components of system (100) by optical sensors (160). Such mirrors may allow optical sensors (160) to view components of system (100) that may not otherwise be within the field of view of sensors (160). Such mirrors may be placed directly adjacent to optical sensors (160). In addition, or in the alternative, such mirrors may be placed adjacent to one or more components of system (100) that are to be viewed by optical sensors (160).

In use of system (100), an operator may select a protocol to run (e.g., from a library of preset protocols), or the user may enter a new protocol (or modify an existing protocol), via user interface (123). From the protocol, controller (121) may instruct the operator which kind of process chip (111) to use, what the contents of vials in reagent storage frame (107) should be, and where to place the vials in reagent storage frame (107). The operator may load process chip (111) into seating mount (115); and load the desired reagent vials and export vials into reagent storage frame (107). System (100) may confirm the presence of the desired peripherals, identify process chip (111), and scan identifiers (e.g., barcodes) for each reagent and product vial in reagent storage frame (107), facilitating the vials to match the bill-of-reagents for the selected protocol. After confirming the starting materials and equipment, controller (121) may execute the protocol. During execution, valves and pumps are actuated to deliver reagents as described in greater detail below, reagents are blended, temperature is controlled, and reactions occur, measurements are made, and products are pumped to destination vials in reagent storage frame (107).

### II. Example of Process Chip

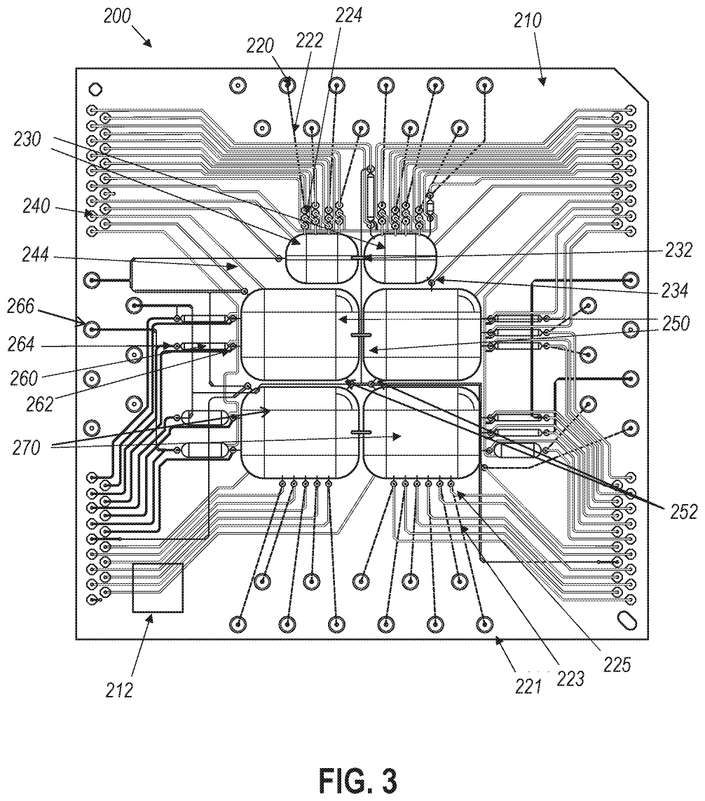

FIG. 3 depicts the example of a process chip (200) in further detail. In combination with the rest of system (100), process chip (200) may be utilized to provide in vitro synthesis, purification, concentration, formulation, and analysis of therapeutic compositions, including but not limited to therapeutic polynucleotides and therapeutic polynucleotide compositions. As shown in FIG. 3, process chip (200) of this example includes a plurality of fluid ports (220). Each fluid port (220) has an associated fluid channel (222) formed in process chip (200), such that fluid communicated into fluid port (220) will flow through the corresponding fluid channel (222). As described in greater detail below, each fluid port (220) is configured to receive fluid from a corresponding fluid line (206) from fluid interface assembly (109). In the present example, each fluid channel (222) leads to a valve chamber (224), which is operable to selectively prevent or permit fluid from the corresponding fluid channel (222) to be further communicated along process chip (200) as will be described in greater detail below.

As also shown in FIG. 3, process chip (200) of this example includes a plurality of additional chambers (230, 250, 270) that may be used to serve different purposes during the process of producing the therapeutic composition as described herein. By way of example only, such additional chambers (230, 250, 270) may be used to provide synthesis, purification, dialysis, compounding, and concentration of one or more therapeutic compositions; or to perform any other suitable function(s). Fluid may be communicated from one chamber (230) to another chamber (230) via a fluidic connector (232). In some versions, fluidic connector (232) is operable like a valve between an open and closed state (e.g., similar to valve chamber (224)). In some other versions, fluidic connector (232) remains open throughout the process of making the therapeutic composition. In the present example, chambers (230) are used to provide synthesis of polynucleotides, though chambers (230) may alternatively serve any other suitable purpose(s).

In the example shown in FIG. 3, another valve chamber (234) is interposed between one of chambers (230) and one of chambers (250), such that fluid may be selectively communicated from chamber (230) to chamber (250). Chambers (250) are provided in a pair and are coupled with each other such that process chip (200) may communicate the fluid back and forth between chambers (250). While a pair of chambers (250) are provided in the present example, any other suitable number of chambers (250) may be used, including just one chamber (250) or more than two chambers (250). Chambers (250) may be used to provide purification of the fluid and/or may serve any of the other various purposes described herein; and may have any suitable configuration. In versions where a chamber (250) is used for purification, chamber (250) may include a material that is configured to absorb selected moieties from a fluidic mixture in chamber (250). In some such versions, the material may include a cellulose material, which may selectively absorb double-stranded mRNA from a mixture. In some such versions, the cellulose material may be inserted in only one chamber (250) of a pair of chambers (250), such that upon mixing the fluid from the first chamber (250) of the pair to the second chamber (250), mRNA and/or some other component may be effectively removed from the fluidic mixture, which may then be transferred to another pair of chambers (270) further downstream for further processing or export. Alternatively, chambers (250) may be used for any other suitable purpose.

Additional valve chambers (252) are interposed between each chamber (250) and a corresponding chamber (270), such that fluid may be selectively communicated from chambers (250) to chambers (270) via valve chambers (252). Chambers (270) are also coupled with each other such that process chip (200) may communicate the fluid back and forth between chambers (270). Chambers (270) may be used to provide mixing of the fluid and/or may serve any of the other various purposes described herein; and may have any suitable configuration.

As shown in FIG. 3, chambers (270) are also coupled with additional fluid ports (221) via corresponding fluid channels (223) and valve chambers (225). Fluid ports (221), fluid channels (223), and valve chambers (225) may be configured an operable like fluid ports (220), fluid channels (222), and valve chambers (224) described above. In some versions, fluid ports (221) are used to communicate additional fluids to chambers (270). In addition, or in the alternative, fluid ports (221) may be used to communicate fluid from process chip (200) to another device. For instance, fluid from chambers (270) may be communicated via fluid ports (221) directly to another process chip (200), to one or more vials in reagent storage frame (107), or elsewhere.

Process chip (200) further includes several reservoir chambers (260). In this example, each reservoir chamber (260) is configured to receive and store fluid that is being communicated to or from a corresponding chamber (250, 270). Each reservoir chamber (260) has a corresponding inlet valve chamber (262) and outlet valve chamber (264). Each inlet valve chamber (262) is interposed between reservoir chamber (260) and the corresponding chamber (250, 270) and is thereby operable to permit or prevent the flow of fluid between reservoir chamber (260) and the corresponding chamber (250, 270). Each outlet valve chamber (264) is operable to meter the flow of fluid between reservoir chamber (260) and a corresponding fluid port (266). In some versions, each fluid port (266) is configured to communicate fluid from a corresponding vial in reagent storage frame (107) to a corresponding reservoir chamber (260). In addition, or in the alternative, each fluid port (266) may be configured to communicate fluid from a corresponding reservoir chamber (260) to a corresponding vial in reagent storage frame (107). In the present example, reservoir chambers (260) are used to provide metering of fluid communicated to and/or from process chip (200). Alternatively, reservoir chambers (260) may be utilized for any other suitable purposes, including but not limited to pressurizing fluid that is communicated to and/or from process chip (200).

As also shown in FIG. 3, process chip (200) of this example includes a plurality of pressure ports (240). Each pressure port (240) has an associated pressure channel (244) formed in process chip (200), such that pressurized gas communicated through pressure port (240) will be further communicated through the corresponding pressure channel (244). As described in greater detail below, each pressure port (240) is configured to receive pressurized gas from a corresponding pressure line (208) from fluid interface assembly (109). In the present example, each pressure channel (244) leads to a corresponding chamber (224, 225, 230, 234, 250, 252, 260, 262, 264, 270) to thereby provide valving or peristaltic pumping via such chambers (224, 225, 230, 234, 250, 252, 260, 262, 264, 270) as described in greater detail below.

Process chip (200) may also include electrical contacts, pins, pin sockets, capacitive coils, inductive coils, or other features that are configured to provide electrical communication with other components of system (100). In the example shown in FIG. 3, process chip (200) includes an electrically active region (212) includes such electrical communication features. Electrically active region (212) may further include electrical circuits and other electrical components. In some versions, electrically active region (212) may provide communication of power, data, etc. While electrically active region (212) is shown in one particular location on process chip, electrically active region (212) may alternatively be positioned at any other suitable location or locations. In some versions, electrically active region (212) is omitted.

Some variations of in a process chip (111, 200) may further include a concentration chamber. In some versions of a concentration chamber, polynucleotides may be concentrated by driving off excess fluidic medium, and the concentrated polynucleotide mixture may be exported out of the concentration chamber for further handling or use. In some variations, the concentration chamber may be in the form of a dialysis chamber. For example, a dialysis membrane may be present within or between plates of process chip (111, 200). In some other variations, a concentration chamber may provide concentration without necessarily serving as a dialysis chamber.

The features of process chip (111, 200) described above are non-limiting examples. Additional features that may be incorporated into a process chip (111, 200) are described in greater detail below. Such additional features may be included in a process chip (111, 200) in addition to, or in lieu of, any of the features described above. There may also be scenarios where a plurality of different kinds of process chips (111, 200) are available to serve different kinds of purposes (e.g., to produce different kinds of therapeutic compositions), such that an operator may select the most appropriate process chip on an ad hoc basis to prepare the desired therapeutic substance. Such selections may be made based on the operator's judgment and/or based on the suggestion or instruction from system (100) via user interface (123). In versions where system (100) suggests the kind of process chip (111, 200) to be used, such suggestion may be based on one or more operator inputs provided via user interface (123) and/or based on other factors.

### III. Manufacture of Therapeutics

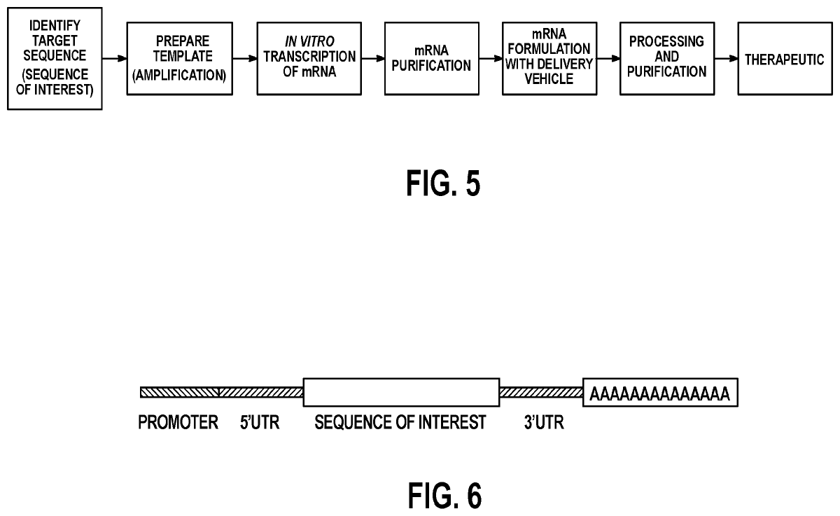

The above-described system may be used for the manufacture of mRNA-based therapeutics as described herein. An example of a method for making an mRNA therapeutic is depicted in FIG. 5. In this example method, a target sequence (“sequence of interest”) is identified. A template comprising the target sequence (“sequence of interest”) may then be prepared and amplified (“amplification”) as shown in FIG. 5. Via in vitro transcription of mRNA, mRNA is manufactured using a template comprising the target sequence. The resulting mRNA comprising the sequence of interest may then be purified and formulated with a delivery vehicle. The purification and the formulation may be carried out on the same process chip as the IVT process, on the same process chip only for these two, or on different process chips. The resulting formulation comprising mRNA may then be further processed and optionally purified for a therapeutic use. Such therapeutics uses may include, for example, cell therapies, oncological treatments, protein replacement, vaccines, expression of effector proteins, inducement of loss of function through expression of dominant negative proteins, and gene/genome editing. In addition to their high potency, mRNA therapeutics also have benefits related to their rapid development cycle, standardized manufacturing, transient expression and low risk of genomic integration. The methods and apparatuses described herein may be used to manufacture mRNA therapeutics for one or more of these categories of therapeutics.

The instant disclosure provides methods by which contamination products in the template preparation/amplification may be improved, in particular, by employing the use of a uracil-containing nucleotide and UNG enzyme in the amplification mixture for use in the amplification of a template as described herein. More specifically, disclosed are methods for manufacturing a polynucleotide therapeutic, particularly an mRNA-based therapeutic (e.g., therapeutic polynucleotide or therapeutic polynucleotide composition), in which contamination resulting from amplification of a polynucleotide sequence is reduced or eliminated.

The disclosed method may be carried out in one or more process chips of the disclosed apparatus. In particular, the disclosed methods may employ an amplification. The method may comprise, for example, one or more of contacting a uracil-DNA glycosylase (UNG) enzyme with an amplification mixture containing a dUTP, modified dUTP, or combinations thereof; inactivating UNG enzyme in the amplification mixture, and amplifying a polynucleotide sequence of interest from a template. The resulting amplified product may be used as an IVT template for performing in vitro transcription (IVT) from the IVT template to produce a therapeutic polynucleotide. The therapeutic polynucleotide may be further processed, including purification and formulation with delivery vehicles, to form a therapeutic polynucleotide composition for use as a therapeutic.

In one aspect, the apparatuses and methods may employ uracil-DNA glycosylase (referred to sometimes as “UNG” or “UDG”, hereinafter referred to as “UNG enzyme”). Uracil-DNA glycosylases (UDGs) are evolutionarily conserved DNA repair enzymes that may remove uracil from DNA. UNG enzyme removes uracil from DNA molecules by cleaving the N-glycosidic bond and initiating the base-excision repair (BER) pathway. Not to be bound by any particular theory, but treatment of uracil-containing polynucleotides containing uracil bases with UNG enzyme is believed to cause cleavage of the glycosidic bond between the deoxyribose of the DNA sugar-phosphate backbone and the uracil base, resulting in subsequent degradation of the uracil-containing polynucleotide. Accordingly, UNG enzyme treatment may be used to minimize, or in some instances eliminate, the presence of uracil-containing polynucleotide that may be found in a contamination product in a process chip that may result from any number of sources, for example, contamination from previous amplification processes, particularly via aerosolization, wherein the previous amplification processes employ one or both of dUTP or modified dUTP.

UNG enzymes for use with the disclosed methods may include any suitable UNG enzyme, and variants thereof, provided such UNG enzyme or variant maintains activity sufficient to cleave uracil from DNA and cause degradation of uracil-containing polynucleotides. On the basis of substrate specificity UDGs are classified into six families, any of which may be used in the disclosed methods. In one aspect, the UNG enzyme may be cod-derived UNG enzyme (cod uracil-DNA N-glycosylase, or “Cod UNG”). Cod UNG enzyme is derived from the Atlantic cod (Gadus morhua) and may be produced in a recombinant E. coli strain containing a modified Cod UNG gene that may be completely and irreversibly heat inactivated.

The methods may further employ the use of dUTP during amplification of the template. For example, using a PCR method, deoxythymidine triphosphate (dTTP) may be replaced with deoxyuridine triphosphate (dUTP), such that amplified product may comprise uracil-containing amplification products. Prior to amplification, a uracil-DNA N-glycosylase (UNG) enzyme may be used to degrade uracil-containing amplification products during an “UNG enzyme incubation period”. During the UNG enzyme incubation period, UNG enzyme is contacted with the amplification mixture, at a concentration and for a time period and temperature sufficient for the desired level of degradation of uracil-containing amplification products. UNG enzyme may be added to the amplification mixture in an amount of from about 0.005 to about 3 U, or from about 0.01 to about 2 U, or from about 0.02 to about 1 U, or from about 0.03 to about 0.5 U, or about 0.01 U UNG enzyme per microliter of final reaction volume. The time period of the UNG enzyme incubation period may vary. For example, in one aspect, the UNG enzyme incubation period may be carried out for about five minutes. In further aspects, the UNG enzyme incubation period may be carried out for about 1 minute to about one hour, or from about 2 minutes to about 45 minutes, or from about 3 minutes to about 30 minutes, or from about 4 minutes to about 15 minutes, or from about 5 minutes to about 10 minutes. Likewise, the temperature used during the UNG enzyme incubation period may vary, and in general, may be a temperature sufficient for the UNG enzyme to exhibit enzymatic activity. For example, the incubation period for degradation of uracil-containing sequences using UNG enzyme may be carried out at room temperature (about 20° C.). In further aspects, the incubation period may be carried out at a temperature of about 10° C. to about 40° C., or about 15° C. to about 30° C.

In general, the UNG enzyme incubation period may be carried out at a time and temperature that allows for 100% degradation of the undesired uracil-containing polynucleotide, or in other aspects, at least about 99%, or at least about 98%, or at least about 97%, or at least about 96%, or at least about 95%, or at least about 94%, or at least about 93%, or at least about 92%, or at least about 91%, or at least about 90% degradation of uracil-containing polynucleotide may be achieved. The degradation of uracil-containing polynucleotides may be facilitated by mixing or other physical or chemical treatment that improves the efficacy or speed of the uracil-containing polynucleotide degradation.

Following the UNG enzyme incubation period described above, during which degradation of uracil-containing polynucleotide is carried out via digestion by contact with UNG enzyme in the amplification mixture, the amplification mixture comprising UNG enzyme may then be subjected to a UNG enzyme inactivation period, during which the UNG enzyme is inactivated. By “inactivation” of UNG enzyme, it is meant in one example herein that the inactivated UNG enzyme does not retain enzymatic activity sufficient to cause degradation of a uracil-containing polynucleotide. UNG enzyme is considered to be substantially inactivated if the inactivation is sufficient to allow for subsequent synthesis and therapeutic integrity of mRNA during the in vitro transcription processes following the UNG enzyme inactivation period. The UNG enzyme inactivation period may result in 100% inactivation of UNG enzyme activity, or at least about 99%, or at least about 98%, or at least about 97%, or at least about 96%, or at least about 95%, or at least about 94%, or at least about 93%, or at least about 92%, or at least about 91%, or at least about 90% inactivation of enzyme activity may be achieved in the inactivation period. In one aspect,

In one aspect, inactivation of UNG enzyme during the UNG enzyme inactivation period may employ applying heat to the amplification mixture containing UNG, such that the heat is sufficient to remove enzymatic activity of the UNG. Heat inactivation may be employed using the thermal controls of the system as described above, though any suitable method of applying heat for inactivation of the UNG enzyme is contemplated herein. In one aspect, the UNG enzyme inactivation period comprises heating the amplification mixture containing UNG enzyme at a temperature of from about 90° C. to about 100° C., or about 95° C. The inactivation period may be carried out for a period of time sufficient to inactivate UNG. In one aspect, the inactivation period may be carried out for about 30 seconds to about 30 minutes, or about 1 minute to about 20 minutes, or from about 2 minutes to about 10 minutes, or about 3 minutes to about 5 minutes. The inactivation time and temperature may vary and may be determined depending on the application; time and temperature may be inversely related, in that a longer inactivation period may be advantageous at lower temperatures, and shorter inactivation periods may be used at higher temperatures. In one aspect, the inactivation period may comprise inactivation by high temperature in an initial PCR cycle, for example, a 98° C., 10 second cycle.

The UNG enzyme inactivation period may alternatively or additionally employ a non-heat inactivation method. For example, inactivation, inactivation of UNG enzyme may be achieved using other methods of inactivation, such as, for example, an inactivating antibody or aptamer (e.g., a short oligonucleotide that binds to and inhibits the UNG enzyme enzyme) in lieu of or in addition to thermal inactivation.

It is noted that the UNG enzyme incubation period, the UNG enzyme activation period, and/or subsequent amplification reactions may be carried out in the initial amplification mixture, or a portion of the amplification mixture, and that such periods and/or reactions may further be carried out in one or more process chips of the disclosed device. The inactivation may be facilitated by mixing or other physical or chemical treatment that improves the efficacy or speed of the uracil-containing polynucleotide degradation.

The amplification mixture of the disclosed apparatuses and methods may comprise a deoxyribonucleotide triphosphate (dNTP) mixture, a DNA polymerase, a template comprising a sequence of interest, and at least one primer pair. The deoxyribonucleoside triphosphate (dNTP) mixture may comprise deoxyribonucleoside triphosphates (dNTPs), such as dATP, dCTP, dUTP, dGTP, dTTP, or derivatives thereof, which may generally be provided in a suitable carrier, such as a buffer. In certain aspects, at least a portion of the total dNTPs of the amplification mixture may comprise deoxyribonucleoside triphosphate (dUTP), such that the amplification process utilizes dUTP in place of dTTP, whether in whole or in part, in the synthesis of uracil-containing polynucleotides. In a further aspect, the dUTPs useful for the disclosed apparatus and methods may include modified dUTPs for example, a 5-substituted dUTP analog, a biotinylated dNTP, 5-modified biotin-16-aminoallyl dUTP, hydroxymethyl dUTP (“dITP”), Biotin-UTP, Digoxigenin-UTP, 2′-F-UTP, biotin-4-dUTP, biotin-11-dUTP, biotin-14-dUTP, biotin-16-AA-dUTP, 5-iodo-dUTP, 5-bromo-dUTP, 5-fluoro-dUTP, 5-propynyl-dUTP, or the like. Modified dUTPs may be used in combination with dUTP to replace dTTP as described in greater detail below.

Any suitable ratios of the dNTP types may be used, depending on the application. One or both of dUTP or a modified dUTP may replace dTTP in whole or in part in the amplification mixture. In one aspect, with respect to the total dTTP added to the amplification mixture, one or both of dUTP or a modified dUTP may replace at least about 10% at least about 20%, at least about 30%, at least about 40% at least about 50%, at least about 60%, at least about 70%, at least about 75%, at least about 80%, at least about 85%, at least about 90%, at least about 95%, or about 100% of the total added dTTP in the amplification mixture. In one aspect, with respect to the total dTTP added to the amplification mixture, one or both of dUTP or a modified dUTP may be provided proportionally in excess of the dNTP concentration for dATP, dCTP, and dGTP, due to the lower efficiency of dUTP incorporation. For example, the concentration of one or both of dUTP or a modified dUTP may be present in an amount that is about 1.5 times, or about 2 times, or about 2.5 times, or about 3 times, or about 3.5 times, or about 4 times, or about 4.5 times, or about 5 times the dNTP concentration for any one concentration of dATP, dCTP, or dGTP. For example, an amplification mixture may comprise about 400 μM dATP, dCTP, dGTP, and about 1200 μM dUTP. In a further example, an amplification mixture may comprise a final concentration of about 0.2 mM dATP, about 0.2 mM dGTP, about 0.2 mM dGTP, and about 0.2-1.0 mM dUTP. In certain aspects, the amplification mixture may be substantially free of dTTP in the amplification mixture. The determination of the amount of dTTP does not include dTTP in the amplification mixture that is in the form of intentionally added template containing a sequence of interest, which will generally take the form of a DNA sequence, which contains dTTP.

As used herein, the term “substantially free” as used with respect to a given substance, includes 100% free of a given substance, or which comprises less than about 0.1%, or less than about 0.5%, or less than about 0.1% of the given substance.

### IV. Template

The methods may include amplification of a template containing the sequence of interest. The template may be a DNA template, for example, linear DNA, plasmid DNA, or combinations thereof. The template may comprise an in vitro transcription facilitator cassette. The in vitro transcription facilitator cassette (IFC) may be an in vitro transcription capable double-stranded DNA. FIG. 6 depicts an example double-stranded DNA template, in which the DNA template comprises a sequence of interest flanked by a 5′UTR sequence and a 3′UTR sequence. The example template comprises the additional expression elements of a promoter region and a poly-A tail.

The template may be incorporated into an in vitro transcription facilitator cassette having functional elements that facilitate in vitro transcription (e.g., from an inserted sequence of interest), such as a promoter, a portion encoding a 5′ untranslated region, (5′UTR), a portion encoding a 3′ untranslated region (3′UTR), and a portion encoding for a polyA tail. The in vitro transcription facilitator cassette may also include one or more linkers useful for cloning a sequence of interest into the in vitro transcription facilitator cassette for expression of the sequence of interest and restriction sites to allow for template linearization.

An in vitro transcription facilitator cassette may be manufactured synthetically or non-synthetically. In one aspect, the in vitro transcription facilitator cassette may be manufactured synthetically. Methods of manufacturing synthetic in vitro transcription facilitator cassette may include using a commercially available DNA synthesizer such as those available from Twist Bioscience (San Francisco, CA) or ThermoFisher Scientific (Waltham, MA). The in vitro transcription facilitator cassette may be assembled from separate pieces of DNA, or may be synthesized as one piece. The in vitro transcription facilitator cassette may be linear and may include compatible ends that may be ligated together. The in vitro transcription facilitator cassette may circular. Circular in vitro transcription facilitator cassettes may include a site (e.g., a restriction endonuclease site) between the portion encoding a polyA region and the promoter configured for generating a linear DNA containing, a promoter, a 5′ UTR, linker region, a 3′UTR, and a portion encoding a polyA region upon application of the appropriate restriction endonuclease. In one aspect, the in vitro transcription facilitator cassette does not encode an antibiotic resistance gene. For example, a synthetically synthesized in vitro transcription facilitator cassette may not require an antibiotic resistance gene, as it is not grown in a biological (e.g., bacterial) cell and does not require antibiotic selection. In one aspect, the in vitro transcription facilitator cassettes do not have an origin of replication (ori) or related control elements for facilitating DNA replication. The total length of the in vitro transcription facilitator cassette may be smaller than many plasmids. In some aspects, the in vitro transcription facilitator cassette may be less than about 2 kb in length, less than about 1.5 kb in length, less than about 1.0 kb in length, less than about 900 bps in length, less than about 800 bps in length, less than about 700 bps in length, or less than about 600 bps in length.

The in vitro transcription facilitator cassette may include a promoter. The enzyme RNA polymerase binds to the promoter and initiates transcription of RNA from a sequence of interest (e.g., after a double stranded DNA template has been assembled from the cassette and the sequence of interest). Examples of promoters useful for transcription in the cassette include natural or modified T7 promoters, natural or modified T3 promoter, or natural or modified SP6 promoters.

The in vitro transcription facilitator cassette may also include a portion encoding an exchangeable 5′ untranslated region (5′ UTR) and a portion encoding an exchangeable 3′ untranslated region (3′ UTR). These regions, which do not themselves get translated into protein or peptide, help regulate translation of an mRNA into a protein or peptide. The in vitro transcription facilitator cassette may also include a portion encoding a polyA tail. A polyA tail in an mRNA is a long chain of tens or hundreds of repeated adenine residues, which is believed to serve several functions such as increasing the stability of the mRNA in the cytoplasm of a cell and aiding in translation of the mRNA into protein. Unlike the rest of the sequence of an mRNA which is encoded directly by the DNA in a template in mRNAs, the polyA tail is not normally directly encoded by the DNA in nature. Rather, naturally occurring DNA contains a shorthand signal, called a polyadenylation signal (e.g., AATAAA), that, along with other DNA sequences, signals the transcription machinery in a cell to add a polyA tail to an mRNA during synthesis. In other words, the length of the polyA tail in naturally occurring mRNAs is determined by the cell that makes mRNA. As seen in FIG. 6, the in vitro transcription facilitator cassette may include a region of DNA that directly encodes for a polyA tail (e.g., the entire tail). The length of the poly A tail is determined by the length (e.g., the number of adenines or poly As or the number of thymidines or polyTs) in the region of DNA that directly encodes for the polyA tail. The region of DNA that direct encodes for the polyA tail may be at least 100 bp long, at least 200 bp long, at least 300 bp long, at least 400 bp long, or at least 500 bp long and may be anything in between these sizes (such as 350 base pairs long). A polyA tail may be added to an mRNA made using the cassette as a template using the same process as used to generate the rest of the mRNA. A transcription mixture may be used to generate the entire mRNA, including the polyA tail, from a double-stranded DNA template as described herein, making a transcription mixture generally free from toxic side products that may otherwise be found in transcription mixtures that are made using cells or cell extracts. The mixture may be safely delivered to a patient with only minimal clean-up. When the double-stranded DNA template is also generated from a mixture substantially free from toxic side products, the transcription product made from the double-stranded DNA template is suitable for direct injection into a patient with only minimal clean-up. Described herein is a double-stranded DNA template that is generated from a well-defined mixture substantially free from toxic side products.

The in vitro transcription facilitator cassette may also include one or more linker regions. The linker region is between the 5′ UTR and the 3′ UTR. The linker region may include at least one cleavable site and generally two cleavable sites. If two or more cleavable sites are present, they may have the same sequence or different sequences. The one or more cleavable restriction sites are useful for inserting a sequence of interest into the in vitro transcription facilitator cassette to generate a synthetic linear or circular ligated product. The sequence of interest is generally inserted between the 5′UTR and 3′UTR in the in vitro transcription facilitator cassette, though in some cases 5′UTR or 3′UTR sequences may be included with the sequence of interest and inserted into the in vitro transcription facilitator cassette along with the sequence of interest. The cleavable site(s) may be a restriction endonuclease site, such as a Type II (type IIG, type IIS) restriction endonuclease, such as BsaI, BbsI, AarI, HhaI, HindIII, NotI, BbvCI, EcoRI, BglII, FokI, AlwI, AcuI, or BcgI available from New England Biolabs (NEB; Ipswich, MA); Promega Corporation (Madison, WI); or ThermoFisher Scientific (Waltham, MA). Suitable restriction sites may vary and will be readily appreciated by one of ordinary skill in the art.

The sequence of interest as described herein may be a short piece of DNA that encodes for a some or all of a product molecule (RNA or protein). The product molecule may or may not comprise functional activity. The sequence of interest may encode a particular protein, a part of a protein, or a particular function. In some cases, it may contain instructions for generating an RNA that does not encode for a particular protein or part of a protein (e.g., it may encode a functional RNA that does not get translated).

A sequence of interest useful for inserting into an in vitro transcription facilitator cassette may be manufactured synthetically or non-synthetically. Methods of manufacturing synthetic genes of interest include by using a commercially available DNA synthesizer and method such as those available from Twist Bioscience (San Francisco, CA) or ThermoFisher Scientific (Waltham, MA). Further, although the sequence of interest may be assembled from separate pieces of DNA, in general it is synthesized as one piece. A sequence of interest may be manufactured as a linear piece of DNA or a circular piece. A circular sequence of interest may be digested with a restriction enzyme to form a linearized sequence of interest. In some aspects, a methylation sensitive restriction enzyme may be used. The manufactured sequence of interest may be purified (e.g., by column, electrophoretic separation, etc.).

A sequence of interest may be cleaved prior to combining it with an in vitro transcription facilitator cassette. In particular, a sequence of interest may be cleaved with the same restriction endonuclease(s) as used to cleave the in vitro transcription facilitator cassette, but may also be generated through enzymatic amplification. In one aspect, the sequence of interest does not encode an antibiotic resistance gene. For example, a synthetically synthesized sequence of interest does not need an antibiotic resistance gene as it is not grown in a biological (e.g., bacterial) cell and does not require antibiotic selection. In general, the sequence of interest does not have an origin of replication (ori) or related control elements for facilitating DNA replication. In some aspects, total length of the sequence of interest may be smaller than many plasmids. The sequence of interest may be less than 2kb in length, less than 1.5 kb in length, less than 1.0 kb in length, less than 900 bps in length, less than 800 bps in length, less than 700 bps in length, or less than 600 bps in length, less than 500 bps in length, less than 400 bps in length, or less than 300 bps in length, less than 200 bps in length, less than 100 bps in length.

Existing DNA templates for performing in vitro transcription and mixtures for performing in vitro transcription may also include crude or semi-purified cellular extracts (e.g., bacterial, other microbial, or other extracts) and may be complex and undefined. Such extracts may include bacterial, other microbial, or other DNA, endotoxin, and/or other undesirable components. When used for generating DNA templates or performing in vitro transcription as part of a process for vaccine or therapeutic use, undesirable components may increase the risk of serious side effects. For example, endotoxin is a large molecule of lipopolysaccharide in the exterior cell wall of Gram-negative bacteria, a commonly used source for generating cellular extracts for use in in vitro transcription reactions. Endotoxin in the bloodstream, such as by injection, may cause a variety of problems in humans and other animals, such as inflammation and sepsis, and may pose a significant health risk. The methods described herein may be useful for making a double-stranded DNA template free of biological contaminants (bacterial, other microbial or other contaminants), free of bacterial (or other microbial or unwanted) DNA, and/or free of endotoxin. The methods described herein may include using defined or synthetic components for making a sequence of interest (which may include a gene of interest), making an in vitro transcription facilitator cassette, and/or making a double-stranded DNA template (or making any intermediaries used for making these materials). The defined or synthetic components may be made from defined or synthetic ingredients such as DNA synthesizers, purified nucleotides and purified enzymes. The defined or synthetic components may be essentially free of bacterial, other microbial, or other DNA, endotoxin, and/or other undesirable components. By avoiding the use of biologically based components, biological contaminants such as DNA and endotoxin do not contaminate the DNA template (or other components) in the first place. Double-stranded DNA templates and downstream materials are safer without the need for difficult or troublesome purification steps. This and other methods herein may include steps of joining a synthetic sequence/gene of interest with a synthetic in vitro transcription facilitator cassette to create a synthetic linear or circular ligated product; removing unreacted synthetic sequence/gene of interest and unreacted synthetic in vitro transcription facilitator cassette; amplifying the circular ligated product to generate a linear, circular or branched amplified DNA; and linearizing the amplified DNA ligated product to generate double stranded DNA template.

As indicated above, joining a sequence/gene of interest with an in vitro transcription facilitator cassette to create a synthetic linear or circular ligated product may include inserting the sequence/gene of interest into an in vitro transcription facilitator cassette as depicted in FIG. 6, which shows a double-stranded DNA template as described herein. The sequence of interest and in vitro transcription facilitator cassette may have the same restriction endonuclease site(s) as described elsewhere herein and the method may include digesting the sequence of interest and the in vitro transcription facilitator cassette with the restriction endonuclease for the restriction endonuclease site, creating compatible ends, and ligating the sequence of interest into the cassette. The method may include combining a sequence of interest, an in vitro transcription facilitator cassette, a restriction endonuclease buffer, a source of energy, one or more restriction endonuclease enzyme(s), a ligase buffer, and a ligase and incubating the mixture for an appropriate amount of time. The buffer(s) may be suitable for or optimized for the particular restriction endonuclease and/or ligase and may be one buffer or may be two (or more) buffers. Endonuclease and/or ligase buffers may be commercially available buffers (e.g., NEB, Promega) and/or may include Tris, potassium, magnesium, sodium chloride, and dithiothreitol, such as Tris-acetate (e.g., 6 mM-90 mM), potassium acetate (50 mM-100 mM), magnesium acetate (5 mM-10 mM), bovine serum albumin (BSA; 50 ug/ml-200 ug/ml) dithiothreitol (1 mM) at a pH from about 7.4 to about 9.0. The ligase may be a commercially available (e.g., NEB, Promega, Thermo Fisher Scientific) or other ligase such as T3 DNA ligase, T4 DNA ligase, or T7 DNA ligase. Digesting may take place from 10 minutes to 4 hours, or any amount of time in between (e.g., 30 min, 1 hour, 2 hours, etc.) The ligating step may take place from 10 minutes to 4 hours, or any amount of time in between (e.g., 30 min, 1 hour, 2 hours, etc.). The digesting and ligating steps may be performed simultaneously or sequentially. A source of energy may be adenosine 5′-triphosphate (ATP) (e.g., from 0.1 mM to 5 mM). Additional quantities of any of the components such as restriction endonuclease and ligase may be added over time and incubation may continue. In some aspects, only materials certified to be animal origin free (“AOF”) will be used for therapeutic manufacturing to reduce the risk of transmitting infectious agents. Some of these methods in which the in vitro transcription facilitator cassette is not circular may include the ligating the ends of the in vitro transcription facilitator cassette and generating a circular in vitro transcription facilitator cassette. Alternatively, other methods for ligating the sequence of interest and the in vitro transcription facilitator cassette such as “chew back” methods may be used. Alternatively or additionally, ligation between the in vitro transcription facilitator cassette and the sequence of interest may be performed using primer extension to generate linear molecules prior to exponential amplification methods.

This or other methods described herein may include removing unreacted synthetic sequence of interest and unreacted synthetic in vitro transcription facilitator cassette away from the synthetic linear or circular ligated product or purifying the double-stranded DNA away from the unreacted synthetic sequence of interest and unreacted synthetic in vitro transcription facilitator. Removing unreacted synthetic sequence of interest and unreacted synthetic in vitro transcription facilitator cassette away from the synthetic circulated ligated product may be done using an enzyme such as an exonuclease (such as exonuclease V) in an appropriate exonuclease buffer (NEB; Promega, Thermo Fisher). The method may include digesting synthetic sequence of interest and unreacted synthetic in vitro transcription facilitator. The method may include passing the digested mixture through a resin or column, such as an ion exchange resin or size exclusion resin, and holding either the unreacted synthetic sequence/gene of interest and unreacted synthetic in vitro transcription facilitator cassette in the column or holding the double-stranded DNA template in the column and allowing the double-stranded DNA template or the unreacted synthetic sequence of interest and unreacted synthetic in vitro transcription facilitator cassette to pass through the resin or column. In some aspects, the method may include one or more of holding digested nucleotides within the resin or column or allowing digested nucleotides to pass through the resin or column, washing and/or eluting the resin or column, holding digested nucleotides within the resin or column, binding unreacted synthetic sequence of interest and unreacted synthetic in vitro transcription facilitator cassette to beads or binding double-stranded DNA to the beads, holding the beads with a magnet and removing either the double-stranded DNA or unreacted synthetic sequence/gene of interest and unreacted synthetic in vitro transcription facilitator cassette from the double-stranded DNA. Resins, columns, and magnetic beads are commercially available, for example from Bangs Laboratories, Inc., (Fishers, IN), Beckman Coulter (Brea, CA), Millipore (Burlington MA), Thermo Fisher, VWR (Radnor, PA).

### V. Amplification

During the amplification process, a sequence of interest is amplified from the template using dUTP, a modified dUTP, or a combination thereof as a substrate, thereby creating an IVT template comprising a uracil-containing polynucleotide sequence corresponding to the sequence of interest. The resulting uracil-containing polynucleotide generated during the amplification process may include dUTP, modified dUTP, and combinations thereof. The amplification process may include amplifying the linear or circular ligated product to generate amplified uracil-containing polynucleotide sequence. Some methods may include amplifying the linear or circular ligated product to generate a linear uracil-containing polynucleotide sequence. Some methods may include amplifying the linear or circular ligated product to generate a linear, branched or circular amplified uracil-containing polynucleotide sequence. Any suitable amplification method may be employed. In one aspect, the amplification process may be a polymerase chain reaction (PCR). Other amplification methods may be used, for example, helicase-dependent amplification (HAD), loop-mediated isothermal amplification (LAMP), multiple displacement amplification (MDA), nucleic acid sequence-based amplification (NASBA), polymerase chain reaction (PCR), rolling circle amplification (RCA), self-sustained sequence replication (3 SR), or strand displacement amplification (SDA). In one example, amplification may be performed entirely within a process chip as described herein.

The amplification may be carried out using the amplification mixture as described above. Temperature and timing of the amplification may be controlled. Amplification may include heating the linear or circular ligated product (e.g., at or above 70° C. to 100° C.) to denature the DNA and then cooling the DNA. The method may include adding a denaturation buffer for denaturing the DNA to the linear or circular ligated product to denature the DNA and then adding a neutralization buffer to the denatured DNA mixture to neutralize the denaturation buffer and leave denatured DNA. The method may include adding an enzyme such as a DNA polymerase enzyme for amplifying or extending the denatured DNA (e.g., Bst DNA polymerase, Φ29 DNA (Phi29) polymerase, Taq DNA polymerase) and amplifying or extending the DNA with the enzyme to generate amplified uracil-containing polynucleotide (e.g., branched, circular, or linear amplified sequence) corresponding to a portion of the DNA sequence provided in the template to form the IVT template, the IVT template being used for in vitro transcription as described herein.

The disclosed methods may include purifying the amplified or extended uracil-containing polynucleotide which forms the IVT template away from the buffer, enzyme, nucleotides, and other unwanted components. The method may include one or more of passing IVT template through beads, resin or a column, such as an ion exchange resin, magnetic beads, or size exclusion resin; holding the IVT template or allowing the IVT template to pass through the beads, resin or column; holding the unwanted enzyme and other components in the beads, resin or column; washing and/or eluting and/or drying and/or rehydrating the resin or column; and repeating one or more of these steps. The method may comprise two or more (a plurality) of depots of beads, resin, or columns and repeating one or more of the washing/eluting/drying/and/or rehydrating steps. The method may further comprise binding DNA to beads, holding the beads with a magnet and removing (washing) unwanted components and contaminants away from the DNA and beads. Resins, columns, and magnetic beads suitable for use are available from Bangs Laboratories, Inc., (Fishers, IN), Beckman Coulter (Brea, CA), Millipore (Burlington MA), Thermo Fisher, and VWR (Radnor, PA).

In some aspects, the IVT template is not linear; it may be branched or circular. Some methods include linearizing IVT template and generating linearized IVT template. Some methods may include adding a restriction endonuclease (in an appropriate buffer) to purified IVT template, incubating the IVT template with the restriction endonuclease, and linearizing the IVT template. The restriction enzyme is chosen to cut outside of the 5′UTR, gene of interest, 3′UTR, and the portion encoding the poly A region. In some aspects, the restriction enzyme may cut between the 3′UTR of one IVT template and the 5′UTR of an adjoining (and downstream) IVT template. The restriction enzyme may be any restriction enzyme, such as a type IIs restriction enzyme as indicated above with regards to restriction enzyme digestion for joining a sequence of interest with a synthetic in vitro transcription facilitator cassette to create a synthetic linear or circular ligated product. In some examples, the restriction enzyme is at least one of BsaI, BbsI, AarI, HhaI, HindIII, NotI, BbvCI, EcoRI, BglII, FokI, AlwI, AcuI, or BcgI available from New England Biolabs (NEB; Ipswich, MA); Promega Corporation (Madison, WI); or ThermoFisher Scientific (Waltham, MA). In some aspects, the restriction endonuclease may be the same restriction endonuclease(s) used for inserting the sequence of interest into the in vitro transcription facilitator cassette. In some aspects, the restriction endonuclease may be different from the restriction endonuclease(s) used for inserting the sequence of interest into the in vitro transcription facilitator cassette.

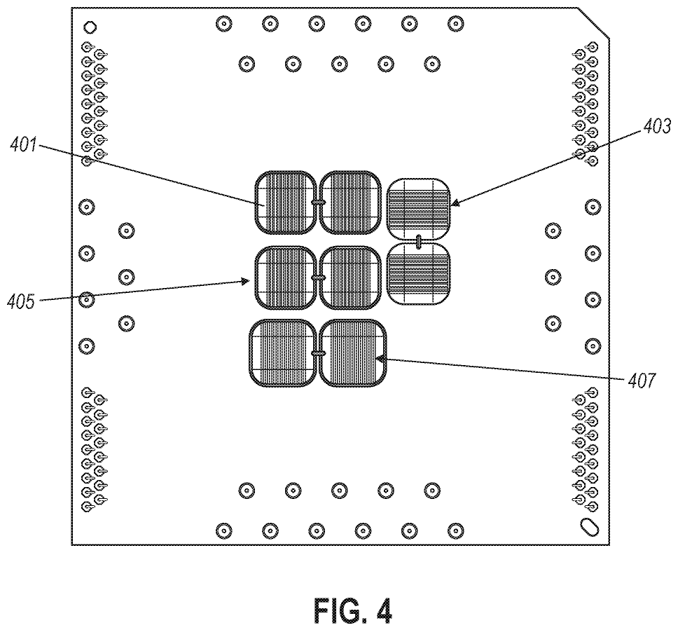

FIG. 4 shows one variation of an architecture of a microfluidic biochip reactor for generating an IVT template. This and other methods described herein may include generating IVT template from the sequence of interest and the in vitro transcription facilitator cassette in a sterile, closed biochip in which all components are sterilely maintained during generation. The sterile, closed biochip is closed to the atmosphere. FIG. 4 depicts a microfluidic biochip reactor with 4 interconnecting reactors (e.g., modules or chambers) through which DNA precursors at different stages along the pathway to becoming a double-stranded DNA template move. For example, in FIG. 4 a ligation reactor (ligation reaction chamber 401), a pre-mixing chamber 403, an amplification reactor (amplification reaction chamber 405) and a digestion reactor (digestion reaction chamber) 407 may be included (connectors and valves are not shown in this example. Different steps of the methods described herein may be carried out in different modules or chambers. The sequence of interest and the in vitro transcription facilitator cassette may be mixed together in the pre-mixing chamber. The sequence of interest and the in vitro transcription facilitator cassette may be joined together to create a ligated product in the ligation reaction chamber. The ligated product may be amplified to generate amplified IVT template in the amplification chamber using methods described herein. The IVT template may be further processed, such as being digested in the digestion reaction chamber to remove unwanted nucleotides or to separate different copies of the amplified sequence of interest.

Because the product of the amplification methods (the IVT template) contains dUTP (and/or a modified dUTP which renders the sequence subject to UNG enzyme degradation, as described above), any potential contamination of the device or process chip of the device by the resulting IVT template may be mitigated or avoided entirely in subsequent runs by employing the disclosed methods, in which UNG enzyme may be used to degrade uracil-containing polynucleotide sequences in the amplification mixture. As such, the system and methods allow for an integrated decontamination step which further allows for high grade therapeutic composition at low risk for contamination events.

### VI. In Vitro Transcription (IVT) Reaction

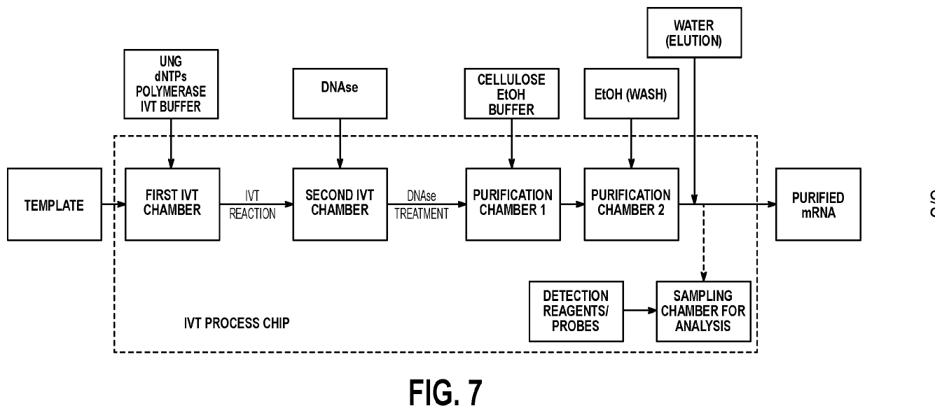

The resulting IVT template, comprising a uracil-containing polynucleotide sequence, may be used for subsequent in vitro transcription reactions to form a therapeutic polynucleotide, more particularly, in vitro transcription (IVT) may be used to produce mRNA. This process may be conducted inside a process chip which may be housed in the process chip control system as described. FIG. 7. depicts an exemplary IVT reaction performed in an IVT process chip, in which the IVT template produced during the amplification process is introduced into a first IVT chamber, the first IVT chamber having reagents for carrying out an amplification reaction, for example, including, but not limited to UNG enzyme, dNTPs (including dUTP, modified dUTP, and combinations thereof), polymerase, and buffer. uracil-containing IVT template is produced from the template, which may be treated with DNAse in the second IVT chamber. The IVT template is purified as described below (using cellulose and an ethanol wash as described), and eluted from Purification Chamber 2. A sampling chamber may be used for analysis of the resulting mRNA, the sampling chamber receiving detection reagents/probes for confirming the content of the resulting RNA. Purified mRNA is obtained from the IVT process chip.

The IVT reaction may involve combining the IVT template with T7 polymerase enzyme, nucleotides and capping reagents and incubating the reaction under controlled conditions to produce capped mRNA molecules. The IVT reactions may take place inside a reaction chamber of a process chip (e.g., an IVT process chip) and process parameters such as temperature, mixing and reagent additions (both at the beginning and throughout the reaction) may be controlled to optimize levels. The process may be driven by the controller, as described above. The buffers and solutions may be delivered via an array of microvalves and volume may be controlled using a pre-set programs that may be specific to the protocol optimized for each mRNA therapeutic product.

Following the IVT reaction, a DNAse treatment may be performed to degrade the template DNA. This may be performed inside the IVT reaction chamber (part of the IVT reactor), and parameters such as dilution rate, enzyme/buffer concentration, temperature and mixing may be controlled to optimized levels. This procedure may be executed autonomously and recorded by a monitoring camera.

### VII. IVT Purification

Described herein are apparatuses and methods for purifying within the process chip. DNAse treated mRNA may be purified to remove impurities and side products. In particular, degraded template, any unreacted nucleotides, enzymes (T7 polymerase and DNAse) and dsRNA affect the quality and immunogenicity of the drug substance. For purification, a 2-step solid-phase reversible immobilization procedure, using supports with different surface chemistries may be used. A cellulose membrane may be used to selectively capture dsRNA under precisely controlled binding conditions and eluting the non-bound fraction into a second purification chamber. The second purification step may use 1-2 um carboxyl-coated paramagnetic beads that selectively capture mRNA greater than 500 bp in length. One or more washes may be performed to remove unbound material, such as nucleotides, enzymes and degraded template. Pure mRNA may then be eluted in USP grade water. In-line microfluidics based purification provides for a fully integrated workflow, without exposing materials to the atmosphere, avoids the use of toxic mobile-phases used with traditional HPLC-based methods and significantly reduces manual intervention.

The disclosed methods and apparatuses may be aseptic methods and apparatuses that permit the manufacture of therapeutic mRNA, or any or all of the components for manufacturing therapeutic mRNAs without exposure to outside atmosphere, and/or to possible sources of RNAse and/or contaminating components that may otherwise be necessary. For example, as described herein these methods may be performed without the addition of bacterial sources of polynucleotides (e.g., in the template), and/or without the addition of components, such as plasticizers, that may be present when purifying via HPLC or other traditional techniques.

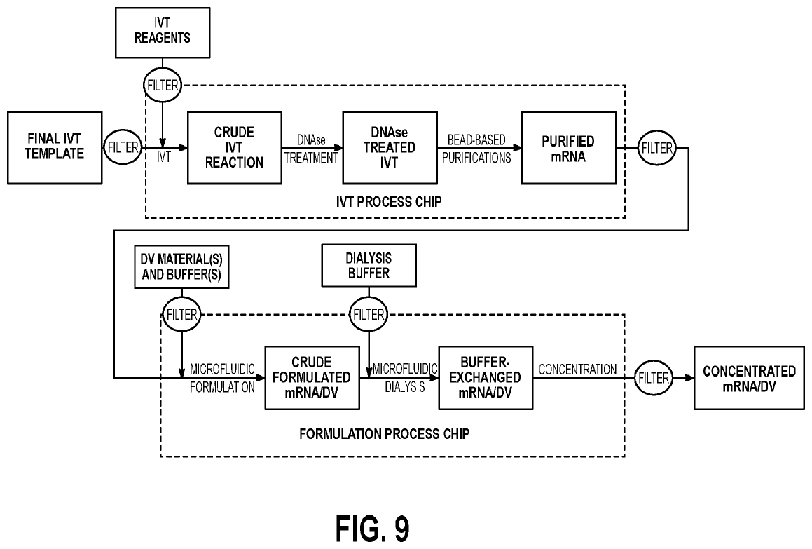

Purified mRNA may be quantified using A260 nm UV absorption, or fluorescence using an mRNA specific fluorescent dye. Additional mRNA QC steps may be performed to confirm purity and identity. The entire mRNA manufacturing process may be conducted inside the process chip control system and reagent addition and export may be performed via the closed-path process chip control system described above, e.g., using aseptic techniques. Finally, a filtration, e.g., through a 0.22 um filter may be performed. The final product may be considered low bioburden drug substance and released for drug product formulation if it meets the acceptance criteria for: yield (e.g., by UV vis/Fluorometry, >6.5 ug mRNA per ul of starting IVT), identity (e.g., by sequencing, 100% consensus homology to target), integrity (e.g., sequencing, <1% mutation rate), purity (e.g., CE, >95% of product in single band), capping efficiency (HPLC, >95% capped mRNA), residual dsRNA (e.g., FRET/Immunoblot, <0.02% (1 ng)), bacterial components (e.g., HCP ELISA (for DNA & protein), <X), bacterial components (e.g., HC-DNA, <X), endotoxin (e.g., LAL test, <0.2 EU/ml), bioburden (e.g., microbial limits testing (MLT)), etc. FIG. 9 depicts an exemplary purification process in an IVT process chip, wherein the final IVT Template and IVT reagents are passed through filters to an IVT process chip where a first, “crude IVT reaction” is carried out to form mRNA. The mRNA is DNAse treated and subjected to a bead-based purification to yield Purified mRNA that may serve as a therapeutic. The resulting purified mRNA may then be filtered and introduced into a formulation process chip. Delivery vehicle materials and buffer are also filtered and introduced into the formulation process chip. During “microfluidic formulation”, a “crude formulated mRNA/DV” composition is formed. Following contact with a filtered dialysis buffer introduced into the formulation process chip, the “crude formulated mRNA/DV” composition” is buffer-exchanged, concentrated, and filtered to yield concentrated mRNA/DV that may be used as a therapeutic.

### VIII. Formulation of mRNA Into Amphipathic Nanoparticles (ANPs)

The purified mRNA may be combined with at least one delivery vehicle molecule (or “delivery vehicle” for short) to form an mRNA nanoparticle. For example, an aqueous solution of mRNA cargo may be combined with an ethanolic solution of delivery vehicle in a microfluidic mixing structure within a formulation process chip. The material may then undergo two post-formulation processing steps involving first an on-chip dialysis process to exchange buffer components in the formulated product, followed by a concentration step to reduce the volume of the drug product to match specifications. The implementation of these processes onto a process chip-based manufacturing device may result in a high degree of control over the formulation process without the need for human intervention and with minimal possibility for human error.

In general, the component parts of the manufacturing methods described herein, including, e.g., synthesizing the IVT template, performing IVT to generate mRNA, purifying the mRNA, combining the mRNA with a delivery vehicle (“DV”) to form a therapeutic composition, dialyzing the therapeutic composition, and/or concentrating the therapeutic composition may be performed on a single process chip and/or multiple process chips, as shown in FIGS. 1-4, described above. Thus, the fluidic path may be continuous or partially continuous (e.g., continuous over the component portion of the manufacturing process, such as one or more of: template formation, IVT template formation, IVT, purifying the mRNA, combining the mRNA with a delivery vehicle to form a therapeutic composition, dialyzing the therapeutic composition, and/or concentrating the therapeutic composition). The same controller apparatus may be used, or different controller apparatuses may be used. The product of each of these component portions may be stored in a fluid vial or depot in the controller apparatus and transferred to a new or subsequent process chip. Thus, in any of these methods and apparatuses, the product may be protected from exposure to the atmosphere.

As noted above, one or more process chips (111, 200, 500) may be utilized to prepare polynucleotide therapeutics (e.g., mRNA therapeutics, etc.). For instance, a polynucleotide such as mRNA in water may be mixed with a delivery vehicle molecule or molecules in ethanol to form complexed nanoparticles. In some scenarios where a process chip (111, 200, 500) is utilized to prepare an mRNA therapeutic composition, it may be desirable to encapsulate mRNA in particles that are on the order of 100 nm in diameter. The process of encapsulating may include tuned mixing of mRNA with delivery vehicle molecules via mixing stages such as the mixing stages (400) described above. Such delivery vehicle molecules may include lipitoid-based molecules, such as amino-lipidated peptoids. During this process, the temperature of the mixing stages (400) may be controlled to a temperature or range of temperatures (e.g., between about 2 degrees C. and about 20 degrees C.) that is calibrated to enhance mixing for mixing in the mixing stages (400). The enhanced mixing temperature may be based on the formulation being mixed (in some examples the sequence of the mRNA and/or the delivery vehicle) within the particular geometry of the mixing chamber.

In some aspects, a peptoid-based lipid formulation may be used as the drug vehicle, which may incorporate both cationic groups and lipid moieties onto an N-substituted peptide (i.e. peptoid) backbone. In one aspect, the delivery vehicle components may be monodisperse, fully-characterizable chemical entities. In one aspect, the delivery vehicle may comprise one or more polyanionic compounds, one or more PEGylated (referring to covalent binding of polyethylene glycol (PEG) molecules) compounds, and one or more cationic compounds. Suitable cationic compounds may include but are not limited to cationic lipids, cationic lipid-peptide conjugates (e.g., lipitoids), cationic peptides, cationic polymers, and lipid-like (lipophilic) cationic compounds. In one aspect, the delivery vehicle may comprise one or more tertiary amino lipidated and/or PEGylated cationic peptide compounds. The tertiary amino lipidated and/or PEGylated cationic peptide compounds may be peptide chains comprising N-substituted amino acid residues.

A controlled and consistent formulation process may be used for maintaining small, uniform particle sizes in mRNA ANP formulations. Delivery vehicle components may be rapidly mixed with mRNA in a controlled ratio by the methods and apparatuses described herein. Exposure of DV components to aqueous solution and interaction between cationic (+) lipids and anionic (−) mRNA may trigger particle formation. This process may be carried out (to control particle size and uniformity) by using the process chips described herein. The mRNA may be dissolved in an acidic buffer (pH 3-5) which may help ensure full protonation of basic functional groups (such as amines) on the delivery vehicle responsible for its cationic charge. The delivery vehicle may be dissolved in an aqueous-miscible organic solvent (typically ethanol) which facilitates the formation of nano-sized particles upon exposure to the aqueous cargo solution. Immediately after mixing, the solution pH may be stabilized by a neutral buffer. The resulting formulation may be stored at 4° C. for weeks with no apparent loss of function. Alternatively, the formulation process may be performed just-in-time and at the point-of-care.

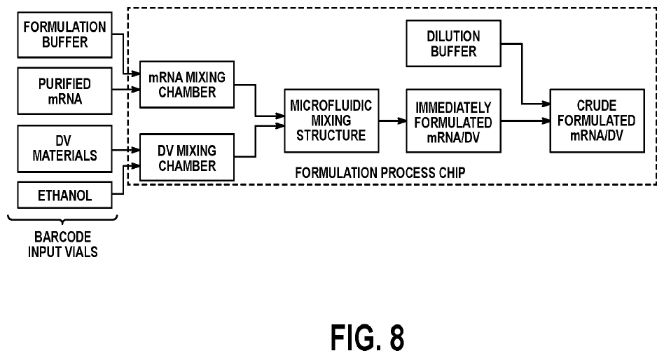

A formulation process chip as described herein may be designed to accomplish these formulation tasks. A first portion of the formulation process chip may include pre-dilution of both the mRNA and the DV components into separate staging chambers. As shown in FIG. 8, the input materials may be advanced from sterile, barcoded vials (e.g., “formulation buffer”, “purified mRNA”, “DV material(s)”, and “ethanol” into the mRNA mixing chamber and the DV mixing chamber. The mRNA material(s) may be pre-diluted in acidic formulation buffer, and the delivery vehicle components may be diluted in ethanol. At this stage, the concentrations of both materials may be adjusted to match specifications for target DV/mRNA ratio, and the volume ratio that matches, e.g., a 3:1 aqueous:ethanol ratio that has previously shown to achieve good mixing behavior. The mRNA mixing chamber and DV mixing chamber contents may be combined in the microfluidic mixing structure as shown in FIG. 8, then immediately formulated into mRNA/DV. Dilution buffer may be added to yield a crude formulated mRNA/DV mixture.

A process chip including a mixing structure may control, with precision, the mixing rate of the material. Faster or slower mixing may be provided, and controlled (e.g., by a controller). For example, a process chip including a mixing structure may provide for a significantly increased delivery vehicle/mRNA mixing rate. At the start of the mixing process, equal pressure may be applied to both mixing chambers which forces fluid through the microfluidic structure at, e.g., 0.5 mL/min. The geometry of this structure may be determined by the rapid mixing time of roughly 3 ms. Under these conditions, amphipathic nanoparticles (ANPs) may be formed as water-insoluble lipid domains on the peptoid molecule are exposed to the aqueous mRNA solution.

In one example, immediately following mixing, ANPs may be diluted with an in-line addition of 1:1 neutral PBS. This may neutralize the acidic formulation buffer and may prepare the formulation for dialysis and concentration. The processes may be controlled through the process chip control system to maintain highly-reproducible particle sizes and formulation properties.

The microfluidic device may allow for the formulation of a personalized therapeutic at the point of care. In Personalized therapeutics may base the therapeutic composition on a specific patient's genetics (e.g., genotype), including generating a specific mRNA composition based on the patient's own sequence). The methods and apparatuses described herein may also or alternatively permit individualized therapeutics. Individualized therapeutics may be based on the patient's phenotype, e.g., based on the category a patient falls into, such as risk factor categories. Individualized therapeutics may therefore adapt specific therapeutics to a patient based on the patient's category. For example, a microfluidic formulation device may allow for multiple mRNAs to be mixed, for example, to generate from a sub-set of mRNAs from a larger library a therapeutic composition that is individualized to a patient based on the components and rations (amounts) of each component which may be determined from phenotype data on the patient. Any of these compositions may be compounded at the point-of-care to generate an optimized treatment for an individual.

### IX. Post-Formulation Processing to Generate Drug Product

Once ANPs are formed during the formulation process, several post-processing operations may be completed on the formulation process chip. These may include dialysis for buffer exchange and ethanol removal, followed by evaporative concentration to reduce volume for dosing.

The resulting nanoparticles may be analyzed on the process chip (e.g., by the process chip control system) for size distribution using, e.g., Dynamic Light Scattering (DLS) and % mRNA encapsulation using fluorescence. Analysis may be completed on a small aliquot of the final formulated material that is diverted from the main fluid path into an optically-transparent sampling chamber. Within this chamber, a fiber-optic light source may be used for the light scattering measurement to determine particle size and dispersity. Next, a fluorescent mRNA-specific probe is used to determine RNA concentration before and after particle disruption by addition of a detergent. This assay may elucidate the mRNA concentration for dosing information and the percentage of mRNA encapsulated in the ANPs versus free in solution. For example, analytical methods that may be used to test the formulated mRNA drug Product may include: Optical clarity (e.g., by visual inspection, no visible, aggregates, clear solution), characterizing lipid composition (e.g., by HPLC), size (e.g., DLS, 80-300 nm), % Encapsulation (e.g., by fluorometry, >95% encapsulation), dispersity (e.g., DLS, PDI<0.25), endotoxin (e.g., LAL test, <0.2 EU/ml), sterility (e.g., culture (USP), <X cfu), pH (e.g., USP, pH 7.4+/−0.2), potency (e.g., bioassay/ELISA, X EC50).

### X. Example of PCR Reaction

The following is a non-limiting example of a PCR reaction which can be used with the disclosed systems and methods.

Cod UNG enzyme (ArcticZymes, Tromsø, Norway) is added to an amplification mixture containing circularized plasmid (containing template for generating specific purified PCR products), and a master mix containing 0.1 u/μL of Q5 PCR enzyme (New England Biolabs, Ipswich, MA) in Q5 reaction buffer (New England Biolabs, Ipswich, MA), 500 μM dATP, dCTP, dGTP, and 2000 μM dUTP (Trilink Biotechnologies, San Diego, CA), and 500 nM mix of forward and reverse primers (Integrated DNA Technologies, Coralville, Iowa) for amplification of a target sequence at a ratio of 0.01 U Cod UNG enzyme per microliter of final reaction volume.

The amplification mixture containing dUTP and UNG enzyme is then incubated at room temperature for 5 minutes prior to target-specific amplification to degrade any potential uracil-containing sequences that may have resulted from prior amplifications or from contamination. Following the incubation period for elimination of contaminants, Cod UNG enzyme is inactivated via a UNG enzyme inactivation period in which the amplification mixture is heated to 95° C. for 2 minutes.

Following the inactivation of UNG, PCR is performed in the process chip, applying the following thermal protocol: 98° C. for 50 seconds, 25 cycles of 95° C. for 10 seconds, 64° C. for 20 s, and 72° C. for 20 seconds. Following the amplification protocol, uracil-containing amplicons containing the sequence of interest are produced, which may then be used for in vitro transcription to produce mRNA, which may then be further purified, concentrated, and combined with delivery vehicle to produce a therapeutic mRNA composition.

It should be appreciated that all combinations of the foregoing concepts and additional concepts discussed in greater detail below (provided such concepts are not mutually inconsistent) are contemplated as being part of the inventive subject matter disclosed herein and to achieve the benefits as described herein. In particular, all combinations of claimed subject matter appearing at the end of this disclosure are contemplated as being part of the inventive subject matter disclosed herein.

1-89. (canceled)

90. A method comprising:
incubating an amplification mixture comprising a uracil-DNA glycosylase enzyme (UNG enzyme), a deoxyribonucleotide triphosphate (dNTP) mixture, a DNA polymerase, a template comprising a sequence of interest, and at least one primer pair;
inactivating said UNG enzyme in said amplification mixture;
amplifying said sequence of interest from said template to form an in vitro transcription (IVT) template, said IVT template comprising a uracil-containing polynucleotide sequence corresponding to said sequence of interest; and
performing in vitro transcription (IVT) from said IVT template to produce a therapeutic polynucleotide;
said deoxyribonucleotide triphosphate (dNTP) mixture comprising a dUTP, a modified dUTP, or combinations thereof.

91. The method of claim 90, said template comprising a DNA template selected from linear DNA, plasmid DNA, and combinations thereof.

92. The method of claim 90, said amplification mixture being substantially free of dTTP.

93. The method of claim 90, said amplification mixture comprising a modified dUTP selected from a 5-substituted dUTP analog, a biotinylated dNTP, 5-modified biotin-16-aminoallyl dUTP, hydroxymethyl dUTP (“dITP”), Biotin-UTP, Digoxigenin-UTP, 2′-F-UTP, biotin-4-dUTP, biotin-11-dUTP, biotin-14-dUTP, biotin-16-AA-dUTP, 5-iodo-dUTP, 5-bromo-dUTP, 5-fluoro-dUTP, 5-propynyl-dUTP, and combinations thereof.

94. The method of claim 90, said therapeutic polynucleotide comprising a therapeutic agent selected from mRNA, a single-stranded mRNA, a circular RNA, a self-replicating RNA, or combinations thereof.

95. The method of claim 90, said contacting; inactivating; and amplifying being performed in a first chamber of a process chip.

96. The method of claim 90, the act of performing in vitro transcription (IVT) being performed in a second chamber of the process chip.

97. An apparatus comprising:
a first chamber to:
incubate a UNG enzyme with an amplification mixture comprising a deoxyribonucleotide triphosphate (dNTP) mixture, a DNA polymerase, a template, and at least one primer pair;
inactivate said UNG enzyme in said amplification mixture;
amplify a polynucleotide sequence of interest from said template to form a uracil-containing polynucleotide; and
a second chamber to:
perform in vitro transcription (IVT) from said uracil-containing polynucleotide to produce a therapeutic polynucleotide.

98. The apparatus of claim 97, the apparatus being part of a process chip.

99. The apparatus of claim 97, the first chamber and second chamber being part of one process chip.

100. The apparatus of claim 97, comprising a controller to drive communication of fluid through the one or more chambers to thereby incubate an amplification mixture until a uracil-containing sequence in said amplification mixture is at least 90% degraded by said UNG enzyme.

101. The apparatus of claim 97, comprising a controller to drive communication of fluid through the one or more chambers to thereby inactivate at least 90% of said UNG enzyme.

102. The apparatus of claim 97, comprising a controller to drive communication of fluid through the one or more chambers such that said inactivating follows said incubating.

103. The apparatus of claim 97, said amplification mixture being substantially free of dTTP.

104. A system comprising:
one apparatus comprising one or more chambers, said apparatus removably inserted into the system; and
a controller to drive communication of fluid through the one or more chambers to thereby:
incubate uracil-DNA glycosylase (UNG) enzyme with an amplification mixture comprising a deoxyribonucleotide triphosphate (dNTP) mixture, a DNA polymerase, a template material, and at least one primer pair;
inactivate said UNG enzyme in said amplification mixture;
amplify a polynucleotide sequence of interest from said template precursor material to form a uracil-containing polynucleotide; and
perform in vitro transcription (IVT) from said uracil-containing polynucleotide to produce a therapeutic polynucleotide.

105. The system of claim 104, said apparatus being part of a process chip.

106. The system of claim 104, said controller to drive communication of fluid through the one or more chambers to thereby incubate uracil-DNA glycosylase (UNG) enzyme with the amplification mixture until a uracil-containing sequence in said amplification mixture is at least 90% degraded by said UNG enzyme.

107. The system of claim 104, said controller to drive communication of fluid through the one or more chambers to thereby inactivate said UNG enzyme in said amplification mixture after incubating uracil-DNA glycosylase (UNG) enzyme with the amplification mixture.

108. The system of claim 104, said controller to drive communication of fluid through the one or more chambers to thereby concentrate said therapeutic polynucleotide.

109. The system of claim 104, said controller to drive communication of fluid through the one or more chambers to thereby combine said therapeutic polynucleotide with a delivery vehicle to form a therapeutic polynucleotide composition.
